(19) United States
(12) Patent Application Publication
(10) Pub. No.: US 2004/0002087 A1
(43) Pub. Date: Jan. 1, 2004

(54) Nucleic acid for detecting endocrine disrupting property of chemical substance, nucleic acid detecting probe and nucleic acid detecting primer containing the nucleic acid, probe-immobilized chip comprising the nucleic acid detecting probe, peptide derived from the nucleic acid and antibody recognizing the peptide, probe-immobilized chip comprising the antibody, and method of detecting endocrine disrupting property of chemical substance using them

(76) Inventors: Eiichi Akahoshi, Kawasaki-shi (JP); Mitsuko Ishihara, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/367,687

(22) Filed: Feb. 19, 2003

(30) Foreign Application Priority Data
Feb. 19, 2002 (JP) 2002-041975
Jan. 20, 2003 (JP) 2003-010742

(51) Int. Cl. C12Q 1/68; C07H 21/04; C12P 19/34

(52) U.S. Cl. 435/6; 536/23.2; 435/91.2

(57) ABSTRACT

The present invention provides a nucleic acid to detect the endocrine disrupting property of a chemical substance, wherein a nucleotide sequence is selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its complementary sequence, a nucleotide sequence described in SEQ ID No. 5 and its complementary sequence, a nucleotide sequence described in SEQ ID No. 14 and its complementary sequence, a nucleotide sequence described in SEQ ID No. 15 and its complementary sequence, a nucleotide sequence described in SEQ ID No. 23 and its complementary sequence, a nucleotide sequence described in SEQ ID No. 27 and its complementary sequence, and a nucleotide sequence described in SEQ ID No. 28 and its complementary sequence.

### CROSS-REFERENCE TO RELATED APPLICATIONS

This application is based upon and claims the benefit of priority from the prior Japanese Patent Applications No. 2002-041975, filed Feb. 19, 2002; and No. 2003-10742, filed Jan. 20, 2003, the entire contents of both of which are incorporated herein by reference.

### BACKGROUND OF THE INVENTION

1. Field of the Invention

The present invention relates to a method for specifically detecting the endocrine disrupting property of a chemical substance, a nucleic acid, a probe and a primer, a probe-immobilized chip, as well as an antibody.

2. Description of the Related Art

Recently, hormone-analogous activity and anti-hormone activity have been found in chemical substances. These chemical substances are called endocrine disrupting substances, or so-called environmental hormones. It has been revealed that endocrine disrupting substances have a toxicity causing a change in the immune system and the nerve system, as well as the reproduction system. Currently, it is believed that 70 or more kinds of chemical substances including dioxin have the endocrine disrupting property. Besides them, there are many chemical substances whose toxicity is unclear. On the other hand, novel chemical substances are being developed every day.

As a method of rapidly detecting the endocrine disrupting property of chemical substances which have been previously used, for example, there is a method using a DNA chip (manufactured by Takarashuzo, IntelliGene™ Human DNA chip for Endocrine Disruption Study). Gene probes used in this kind of DNA chip are the known gene groups selected from an intranuclear receptor gene group, a transcriptional cofactor gene group, a gonad differentiation gene group, and a gene group involved in cell cycle. However, these gene groups are not genes which specifically respond to the endocrine disrupting property of chemical substances. For that reason, by the previous method, it is difficult to specifically detect the endocrine disrupting property of chemical substances.

### BRIEF SUMMARY OF THE INVENTION

According to a first aspect of the present invention, there is provided a nucleic acid to detect the endocrine disrupting property of a chemical substance, wherein a nucleotide sequence is selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its complementary sequence, a nucleotide sequence described in SEQ ID No. 5 and its complementary sequence, a nucleotide sequence described in SEQ ID No. 14 and its complementary sequence, a nucleotide sequence described in SEQ ID No. 15 and its complementary sequence, a nucleotide sequence described in SEQ ID No. 23 and its complementary sequence, a nucleotide sequence described in SEQ ID No. 27 and its complementary sequence, and a nucleotide sequence described in SEQ ID No. 28 and its complementary sequence.

According to a second aspect of the present invention, there is provided a nucleic acid detecting probe comprising the nucleic acid as defined in the first aspects.

According to a third aspect of the present invention, there is provided a nucleic acid detecting primer comprising the nucleic acid as defined in the first aspects.

According to a fourth aspect of the present invention, there is provided a probe-immobilized chip comprising a substrate, and a nucleic acid detecting probe as defined in the second aspect, the nucleic acid detecting probe being solid-phased on the substrate.

According to a fifth aspect of the present invention, there is provided a method of detecting the endocrine disrupting property of a chemical substance, comprising:

(a) making the chemical substance act on a specimen;

(b) after the chemical substance has acted, obtaining a specimen nucleic acid from the specimen;

(c) making the specimen nucleic acid react with the nucleic acid detecting probe as defined in the second aspect;

(d) obtaining an extent of expression of the target nucleic acid by detecting hybridization between a nucleic acid detecting probe and a target nucleic acid; and

(e) detecting the endocrine disrupting property of the chemical substance by comparing an extent of expression of the target nucleic acid obtained in (d) with an extent of expression of the target nucleic acid when the compound has not acted on a specimen.

According to a sixth aspect of the present invention, there is provided a method of detecting the endocrine disrupting property of a chemical substance, comprising:

(a) making the chemical substance act on a specimen;

(b) after the chemical substance has acted, obtaining a specimen nucleic acid from the specimen;

(c) obtaining an amplified product of the specimen nucleic acid using 2 or more kinds of nucleic acid detecting primers as defined in the third aspect and a nucleic acid amplifying enzyme;

(d) obtaining an extent of expression of the target nucleic acid by analyzing the amplified product obtained in (c); and

(e) detecting the endocrine disrupting property of the chemical substance by comparing an extent of expression of the target nucleic acid obtained in (d) with an extent of expression of the target nucleic acid when the compound has not acted on a specimen.

According to a seventh aspect of the present invention, there is provided a peptide encoded by the nucleic acid as defined in any one of the nucleic acid described in SEQ ID No. 1, SEQ ID No. 3, SEQ ID No. 14, SEQ ID No. 15 and SEQ ID No. 23.

According to an eighth aspect of the present invention, there is provided an antibody recognizing the peptide as defined in the seventh aspect.

According to a ninth aspect of the present invention, there is provided a probe-immobilized chip comprising a substrate, and at least one probe selected from a group consisting of the antibody as defined in the eighth aspect which are solid-phased on the substrate.

According to a tenth aspect of the present invention, there is provided a method of detecting the endocrine disrupting property of a chemical substance, comprising:

(a) making the chemical substance act on a specimen;

(b) after the chemical substance has acted, obtaining a specimen sample from the specimen;

(c) making the specimen sample react with at least one detecting probe selected from a group consisting of the antibody as defined in the eighth aspect;

(d) detecting the presence of the target substance by detecting binding of the detecting probe with a target substance; and

(e) detecting the endocrine disrupting property of the chemical substance by comparing the detection results obtained in (d) with the results of detection of the presence of the target substance when the compound has not acted on a specimen.

### BRIEF DESCRIPTION OF THE SEVERAL VIEWS OF THE DRAWING

FIG. 1 is a schematic view showing the results of electrophoresis performed in Example 1;

FIG. 2 is a schematic view showing the results of electrophoresis performed in Example 1;

FIG. 3 is a schematic view showing the results of electrophoresis performed in Example 3;

FIG. 4 is a view showing one Example of a probe-immobilized substrate in accordance with an aspect of the present invention; and

FIG. 5 is a view showing one examples of a probe-immobilized substrate in accordance with an aspect of the present invention.

### DETAILED DESCRIPTION OF THE INVENTION

As used herein, the term “endocrine disrupting substance” refers to an exogenous chemical substance which inhibits or promotes various processes such as the homeostasis of the living body, and synthesis, storage, secretion, internal transport, receptor binding, hormone activity and excretion of various internal hormones involved in reproduction, development and behavior, and is also a term which is also named an exogenous endocrine disrupting substance, an endocrine disrupting substance, an endocrine disrupting chemical substance, an endocrine disorder substance or an environmental hormone. In addition, in the present specification, the terms “exogenous endocrine disrupting substance”, “endocrine disrupting substance”, “endocrine disrupting chemical substance”, “endocrine disorder substance” and “environmental hormone” are used interchangeably.

As used herein, the term “endocrine disrupting property” refers to the activity in which the original homeostasis of cells preserved by hormones is disrupted by disrupting the activity of endocrine hormones. For example, an endocrine disrupting substance acts on a receptor for an endocrine hormone present in a cellular nucleus, whereby, disrupts the activities of endocrine hormones which originally act on the receptor.

I. Genes Whose Expressed Amount is Specifically Influenced by the Endocrine Disrupting Property

According to one aspect of the present invention, there is disclosed a nucleic acid including a gene whose expressed amount in a specimen is specifically influenced by the endocrine disrupting property.

Regarding the case where 2,3,7,8-tetrachloro-benzo-p-dioxin (hereinafter, referred to as TCDD) acts on mouse neuroblastoma Neuro2a in the presence of triiodetyronine, and the case where TCDD did not act on the aforementioned cell, the present inventors compared the RNAs expressed in respective cells by the differential display method. As a result, a gene whose expressed amount is specifically influenced by TCDD, was revealed.

As used herein, Neuro2a is a cell which is sensitive to the endocrine hormone. That is, under the conditions where an endocrine hormone is present in a culturing system for the cell, expression of various cell functions is controlled by government by the hormone and, under the conditions where no endocrine hormone is present in the culturing system, Neuro2a is a cell whose various cell functions is not expressed. Therefore, when an endocrine disrupting substance acts on Neuro2a while triiodotyronine is present as an endocrine hormone, it is possible to load only the activity of the endocrine disrupting property of the aforementioned endocrine disrupting substance on the aforementioned cell. Therefore, by analysis under such conditions, a gene whose expression is specifically influenced by the endocrine disrupting property was revealed.

A nucleic acid including a gene whose expressed amount is reduced specifically by the endocrine disrupting property identified by the present inventors is ND83-3 represented by a nucleotide sequence described in SEQ ID No. 3. This shows 94% homology with the clone name IMAGE:3496023 (Database Accession No. BC007484, American Type Culture Collection (ATCC) Accession No. 5670434).

In addition, a further nucleic acid identified by the present inventors is ND83-4 represented by a nucleotide sequence described in SEQ ID No. 5. Clones exhibiting 85% or more homology with this nucleotide sequence are the following six clones, RIKEN:G431004A07 (Database Accession No. BB795235), IMAGE:3156947 (Database Accession No. AW910492, ATCC Accession No. 5670434), IMAGE:540238 (Database Accession No. AI427138, ATCC Accession No. 997352), IMAGE:805651 (Database Accession No. AI467121, ATCC Accession No. 1067917), UI-M-CD1-azs-c-11-0-UI (Database Accession No. BE953230), and mouse RP24-388P13 (Database Accession No. 101915).

Deposit Authority and Deposit Accession No. of Clone

| | | Deposit |
|---|---|---|
| Clone Name | Deposit Authority | Accession No. |
| ND83-3 | | |
| IMAGE:3496024 | American Type Culture | ATCC Number: |
| | Collection (ATCC) | 5670434 |
| ND83-4 | | |
| RIKEN:G431004A07 | Unkown | Unkown |
| IMAGE:3156947 | American Type Culture | ATCC Number: |
| | Collection (ATCC) | 5386654 |
| IMAGE:540238 | American Type Culture | ATCC Number: |
| | Collection (ATCC) | 997352 |
| TMAGE:8005651 | American Type Culture | ATCC Number: |
| | Collection (ATCC) | 1067917 |
| UI-M-CD1-azs-c-11-0-UI | Research genetics, inc | Unkown |
| RP24-388P13 | Unkown | Unkown |

A further nucleic acid identified by the present inventors is ND118-1 represented by a nucleotide sequence described in SEQ ID No. 14. This nucleotide sequence is encoded in a region of 107213067 to 107213304 on mouse 11^{th} chromosome.

In addition, a further nucleic acid identified by the present inventors is ND818-2 represented by a nucleotide sequence described in SEQ ID No. 27. This is encoded in a region of 105476953 to 1054477260 on mouse 3^{rd} chromosome, and a mouse hypothetical protein MGC:7720 (Accession No. NM030249) is encoded in the neighborhood of the same region.

In addition, a further nucleic acid identified by the present inventors is ND87-3 represented by a nucleotide sequence described in SEQ ID No. 28. This is encoded in a region of 7646283 to 7646436 on mouse 10^{rd} chromosome, and a function unknown gene (Accession No. AK014406), anticipated by Ensembl, is encoded in the same region.

An extent of expression of genes indicated by nucleotide sequences of ND83-3, ND83-4, ND118-1, ND818-2 and ND87-3 is influenced by a chemical substance having the endocrine disrupting property. Therefore, by analyzing an extent of expression of genes indicated by nucleotide sequences of ND83-3, ND83-4, ND118-1, ND818-2 and ND87-3, it is possible to analyze the presence or absence of the endocrine disrupting property and an extent of the endocrine disrupting property regarding a chemical substance, for which the endocrine disrupting property is unknown. Accordingly, a nucleic acid provided as one aspect of the present invention, a nucleic acid represented by the similar nucleotide sequence to that of ND83-3, ND83-4, ND118-1, ND818-2 and ND87-3 can be used as a marker for the endocrine disrupting property.

A nucleic acid for detecting the endocrine disrupting property of a chemical substance which can be provided as one aspect of the present invention is a nucleic acid for detecting the endocrine disrupting property of a chemical substance, and may be a nucleic acid represented by a nucleotide sequence selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its complementary sequence, a nucleotide sequence described in SEQ ID No. 5 and its complementary sequence, a nucleotide sequence described in SEQ ID No. 14 and its complementary sequence, a base sequence described in SEQ ID No. 15 and its complementary sequence, a nucleotide sequence described in SEQ ID No. 23 and its complementary sequence, a nucleotide sequence described in SEQ ID No. 27 and its complementary sequence, and a nucleotide sequence described in SEQ ID No. 28 and its complementary sequence.

Here, a nucleotide sequence described in SEQ ID No. 3 is a sequence including 1969 position to 2509 position in a nucleotide sequence of SEQ ID No. 1. Further, a nucleotide sequence described in SEQ ID No. 5 is a sequence including 269 position to 678 position of a nucleotide sequence of SEQ ID No. 4.

In addition, a nucleic acid for detecting the endocrine disrupting property of a chemical substance may be a nucleic acid represented by a nucleotide sequence selected from a group consisting of a nucleic acid sequence having 85% to 100% homology with a nucleotide sequence described in SEQ ID No. 1 and its complementary sequence, a nucleic acid sequence having 85% to 100% homology with a nucleotide sequence described in SEQ ID No. 3 and its complementary sequence, a nucleic acid sequence having 85% to 100% homology with a nucleotide sequence described in SEQ ID No. 4 and its complementary sequence, a nucleic acid sequence having 85% to 100% homology with a nucleotide sequence described in SEQ ID No. 5 and its complementary sequence, a nucleic acid sequence having 85% to 100% homology with a nucleotide sequence described in NEQ ID No. 14 and its complementary sequence, a nucleic acid sequence having 85% to 100% homology with a nucleotide sequence described in SEQ ID No. 15 and its complementary sequence, a nucleic acid having 85% to 100% homology with a nucleotide sequence described in SEQ ID No. 23 and its complementary sequence, a nucleic acid having 85% to 100% homology with a nucleotide sequence described in SEQ ID No. 27 and its complementary, and a nucleic acid having 85% to 100% homology with a nucleotide sequence described in SEQ ID No. 28 and its complementary.

A nucleic acid for detecting the endocrine disrupting property of a chemical substance which can be provided as another aspect of the present invention may be a nucleic acid comprising consecutive 15 bases to 30 bases contained in a nucleotide sequence selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its comple-mentary sequence, a nucleotide sequence described in SEQ ID No. 5 and its complementary sequence, a nucleotide sequence described in SEQ ID No. 14 and its complementary sequence, a nucleotide sequence described in SEQ ID No. 15 and its complementary sequence, a nucleotide sequence described in SEQ ID No. 23 and its complementary sequence, a nucleotide sequence described in SEQ ID No. 27 and its complementary sequence as well as a nucleotide sequence described in SEQ ID No. 28 and its complementary sequence.

In addition, a nucleic acid for detecting the endocrine disrupting property of a chemical substance may be a nucleic acid comprising consecutive 15 bases to 30 bases contained in a nucleotide sequence selected from a group consisting of a nucleic acid sequence having 85% to 100% homology with a nucleotide sequence described in SEQ ID No. 1 and its complementary sequence, a nucleic acid sequence having 85% to 100% homology with a nucleotide sequence described in SEQ ID No. 3 and its complementary sequence, a nucleic acid sequence having 85% to 100% homology with a nucleotide sequence described in SEQ ID No. 4 and its complementary sequence, a nucleic acid sequence having 85% to 100% homology with a nucleotide sequence described in SEQ ID No. 5 and its complementary sequence, a nucleic acid sequence having 85% to 100% homology with a nucleotide sequence described in SEQ ID No. 14 and its complementary sequence, a nucleic acid sequence having 85% to 100% homology with a nucleotide sequence described in SEQ ID No. 15 and its complementary sequence, a nucleic and sequence having 85% to 100% homology with a nucleotide sequence described in SEQ ID No. 23 and its complementary sequence, a nucleic acid sequence having 85% to 100% homology with a nucleotide sequence described in SEQ ID No. 27 and its complementary sequence, and a nucleic and sequence having 85% to 100% homology with a nucleotide sequence described in SEQ ID No. 28 and its complementary sequence.

As used herein, the term “nucleic acid” refers to various naturally occurring DNAs and RNAs, as well as artificially synthesized nucleic acid analogs such as peptide nucleic acid, morpholino nucleic acid, methylphosphonate nucleic acid and S-oligo nucleic acid.

As used herein, the term “having 85% to 100% homology” indicates that two nucleotide sequences are homologous to each other by 85% to 100%, and the remaining 0% to 25% of a nucleotide sequence is a part having no homology by modification by deletion of 1 or more bases, and/or substitution of 1 or more bases, and/or addition of 1 or more bases.

Here, “N” or “n” in each sequence may be any nucleotide of adenine, thymine, guanine or cytosine.

II. Nucleic Acid Detecting Probe

According to one aspect of the present invention, the aforementioned nucleic acid for detecting the endocrine disrupting property of a chemical substance or a part thereof can be used as a nucleic acid detecting probe for detecting the endocrine disrupting property of a chemical substance. Such nucleic acid detecting probe for detecting the endocrine disrupting property of a chemical substance is within the scope of the present invention.

In accordance with the present invention, a preferable nucleic acid detecting probe for detecting the endocrine disrupting property of a chemical substance may be a nucleic acid detecting probe containing a nucleic acid comprising consecutive 15 bases to 30 bases contained in a nucleotide sequence selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its complementary sequence, a nucleotide sequence described in SEQ ID No. 5 and its complementary sequence, a nucleotide sequence described in SEQ ID No. 14 and its complementary sequence, a nucleotide sequence described in SEQ ID No. 15 and its complementary sequence, a nucleotide sequence described in SEQ ID No. 23 and its complementary sequence, a nucleotide sequence described in SEQ ID No. 27 and its complementary sequence, and a nucleotide sequence described in SEQ ID No. 28 and its complementary sequence.

In addition, a nucleic acid detecting probe for detecting the endocrine disrupting property of a chemical substance may contain a nucleic acid comprising consecutive 15 bases to 30 bases contained in a nucleotide sequence selected from a group consisting of a nucleic acid sequence having 85% to 100% homology with a nucleotide sequence described in SEQ ID No. 1 and its complementary sequence, a nucleic acid sequence having 85% to 100% homology with a nucleotide sequence described in SEQ ID No. 3 and its complementary sequence, a nucleic acid sequence having 85% to 100% homology with a nucleotide sequence described in SEQ ID No. 4 and its complementary sequence, a nucleic acid sequence having 85% to 100% homology with a nucleotide sequence described in SEQ ID No. 5 and its complementary sequence, a nucleic acid sequence having 85% to 100% homology with a nucleotide sequence described in SEQ ID No. 14 and its complementary sequence, a nucleic acid sequence having 85% to 100% homology with a nucleotide sequence described in SEQ ID No. 15 and its complementary sequence, a nucleic acid sequence having 85% to 100% homology with a nucleotide sequence described in SEQ ID No. 23 and its complementary sequence, a nucleic acid sequence having 85% to 100% homology with a nucleotide sequence described in SEQ ID No. 27 and its complementary sequence, and a nucleic acid sequence having 85% to 100% homology with a nucleotide sequence described in SEQ ID No. 28 and its complementary sequence.

Still further, preferable examples of a nucleic acid detecting probe for detecting the endocrine disrupting property of a chemical substance include nucleic acids represented by nucleotide sequences described in SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 17, SEQ ID No. 18, SEQ ID No. 19, SEQ ID No. 20, SEQ ID No. 21, SEQ ID No. 22, SEQ ID No. 25 and SEQ ID No. 26.

In accordance with one aspect of the present invention, a nucleic acid detecting probe for detecting the endocrine disrupting property of a chemical substance may contain a desired label substance. In addition, a nucleic acid detecting probe for detecting the endocrine disrupting property of a chemical substance may contain a further sequence in addition to the aforementioned nucleic acids.

III. Method for Detecting Endocrine Disrupting Property Using Nucleic Acid Detecting Probe

By using such nucleic acid detecting probe, it is possible to detect the endocrine disrupting property of a chemical substance by the following procedures: First, a chemical substance which is a test subject is made to act on a specimen. Then, a specimen nucleic acid is prepared from the specimen. The resulting specimen nucleic acid and the aforementioned nucleic acid detecting probe are reacted. When a target sequence is contained in a specimen nucleic acid, hybridization occurs. Subsequently, the presence or absence and an extent of hybridization are detected. This detection of this binding can be performed, for example, by performing hybridization using a nucleic acid detecting probe which has been labeled with a detectable labeling substance in advance, and detecting the labeling substance after hybridization.

Further, determination of the presence or absence and an extent of the endocrine disrupting property of a chemical substance which is a test subject, can be performed by comparing the results obtained by using a control nucleic acid taken from a specimen which has been treated under the same conditions except that the above chemical substance has not acted. Alternatively, the presence or absence of the endocrine disrupting property of a chemical substance which is a test subject may be determined by setting a threshold in advance by comparing the results in such control nucleic acid, and determining whether or not it exceeds the threshold. Such method of detecting the endocrine disrupting property of a chemical substance is provided as another aspect of the present invention.

As used herein, the term “specimen” refers to the living substance on which a chemical substance of a test subject has acted. For example, the specimen may be organism individuals such as a mouse, rat, cat, dog, cow, sheep, pig, sheep and monkey, or may be cultured cells and tissues derived from animal living body including a human being.

In addition, when organism individuals are used as a specimen, a specimen nucleic acid may be prepared from blood, serum, lymph liquid and tissue obtained from individuals. In that case, if necessary, necessary arbitrary pretreatment such as homogenization and extraction may be performed. Such pretreatment can be selected by a person skilled in the art depending on a sample which is a subject. In addition, when a specimen is a cultured cell or tissue, pretreatment may be performed similarly and, thereafter, a specimen nucleic acid may be extracted, or extraction may be performed without pretreatment.

A specimen nucleic acid may be prepared from a specimen to be used by the means known per se. As used herein, the term “specimen nucleic acid” generally refers to mRNA or whole RNA expressed in a subject. A step of obtaining a nucleic acid to be tested from a subject can be performed by the means known per se. For example, a commercially available kit may be employed, or a solid-liquid extracting method using a carrier such as an oligo dT column may be employed, for example, a liquid-liquid extracting method such as a phenol-chloroform method and the like may be employed, being not limiting.

Moreover, when an amount of a specimen nucleic acid is small, if necessary, an operation of amplifying a polynucleotide may be performed. An amplifying operation may be performed, for example, by a reverse transcription polymerase chain reaction (RT-PCR) or a polymerase chain reaction (hereinafter, abbreviated as PCR).

As used herein, the term “target sequence” is a sequence which is complementary to a nucleic acid for detecting the endocrine disrupting property of a chemical substance or a part thereof in accordance with the present invention, and is a sequence which can hybridize with a nucleic acid detecting probe in accordance with the present invention.

In addition, as a primer used in the aforementioned amplifying operation, a nucleic acid detecting primer of the present invention described later may be employed.

IV. Probe-Immobilized Chip

The aforementioned nucleic acid detecting probe in accordance with the present invention may be provided as a probe-immobilized chip which is immobilized on a substrate. Further, such probe-immobilized chip is included in the scope of the present invention.

According to another aspect of the present invention, the probe-immobilized chip can be made by immobilizing the aforementioned nucleic acid detecting probe on a substrate as described later. Examples of the substrate used in accordance with the present invention include substrates such as a porous body, a microtiter plate, a bead, a spherical substance, a particulate substance, a magnetic body and a magnetic bead and the like. In addition, a material, a size and a shape of a substrate on which a nucleic acid detecting probe is to be immobilized are not particularly limited.

Detection of a nucleic acid sequence in a sample substance employing the probe-immobilized chip can be performed, for example, by extracting a nucleic acid component from a sample substance taken from a specimen, contacting the aforementioned sample nucleic acid with a probe-immobilized chip, and detecting a hybridization reaction between a probe on a probe-immobilized chip and a sample nucleic acid.

In order to detect a hybridization reaction between a probe on the probe-immobilized chip and a nucleic acid component in a sample, two kinds of (1) a method using a label substance, and (2) an electrochemical method are mainly contemplated.

In the case of (1) method using a label substance, a sample nucleic acid may be labeled in advance with a fluorescent pigment such as FITC, Cy3, Cy5 and rhodamine, or an enzyme such as biotin, hapten, oxidase and phosphatase, or an electrochemically active substance such as ferrocene and quinine. Alternatively, detection may be performed by using a second probe labeled with the aforementioned substance. A plurality of labeling substances may be used at the same time.

In the case of (2) electrochemical method, an electrically conductive substance is used as a substrate on which a probe is to be immobilized, and a probe-immobilized chip is used as an electrode. Detection of the presence of a hybridization reaction using this electrode may use a counter electrode and a reference electrode in addition to this electrode like other general electrochemical detecting methods. When a reference electrode is arranged, for example, general reference electrodes such as a silver/silver chloride electrode and a mercury/mercury chloride electrode may be employed. Probes having different nucleotide sequences may be immobilized on different separate substrates, respectively, to construct a chip disposed on the same substrate. Thereby, high-precision measurement is possible. In this case, to which probe an electrochemical signal obtained from each electrode corresponds is detected.

It is desirable that a hybridization reaction between a nucleic acid component extracted from a sample substance and a probe immobilized on a probe-immobilized chip is performed, for example, as follows: That is, a hybridization reaction is performed in a buffer having an ionic strength in a range of 0.01 to 5 and pH in a range of 5 to 10. To this solution, dextran sulfate which is a hybridization promoter, as well as a salmon spermatozoon DNA, a bovine thymus DNA, EDTA and a surfactant may be added. An extracted nucleic acid component may be added thereto, and may be thermally degenerated at 90° C. or higher. Insertion of a probe-immobilized chip may be performed immediately after degeneration or after rapid cooling to 0° C. Alternatively, a hybridization reaction may be performed by adding dropwise a liquid on a substrate. During a reaction, a reaction rate may be heightened by operations such as stirring and shaking. A reaction temperature is, for example, in a range of 10° C. to 90° C., and a reaction time is approximately 1 minute to overnight. After a hybridization reaction, an electrode is removed and washed. Washing may be performed using a buffer having an ionic strength in a range of 0.01 to 5 and pH 5 to 10.

In the case where (1) a labeling substance is used, detection of a hybridization may be performed by using a suitable detecting apparatus depending upon a kind of a label and detecting a label in a labeled nucleotide sequence in a sample or in a secondary probe. When a label is a fluorescent substance, for example, a label may be detected using a fluorescent detector.

In the case of (2) electrochemical method, detection may be performed by the following procedures: After a substrate is washed, a double-stranded chain-recognizing body which selectively binds to a double-stranded chain part formed on the surface of an electrode, is acted, and an electrochemical measurement is performed. A double-stranded-recognizing body used herein is not particularly limited, but for example, Hoechst 33258, acridine orange, quinacrine, dounomycin, metallointercalator, bisintercalator such as bisacridine, trisintercalator and polyintercalator can be used. Further, these intercalators may be modified with an electrochemically active metal complex, for example, ferrocene, biorogen and the like. The concentration of a DNA binding substance is different depending on a kind thereof, but generally, the substance is used in a range of 1 ng/mL to 1 mg/mL. Upon this, a buffer having pH in a range of 5 to 10 may be used in an ionic strength of 0.001 to 5. An electrode is reacted with a double-stranded chain-recognizing body, and washed, and an electrochemical measurement may be performed.

In the electrochemical measurement, potential equal to or greater than potential at which a double-stranded chain-recognizing body reacts may be applied, and a reaction current value derived from a double-stranded chain-recognizing body may be measured. Upon this, potential can be scanned at a constant rate, or can be applied by pulse, or constant potential can be applied. In measurement, current and voltage may be controlled, for example, using an apparatus such as a potentiostat, a digital multimeter and a function generator. Based on the resulting current value, the concentration of a target nucleic acid can be calculated from a calibration curve.

A nucleotide sequence detecting apparatus using an electrode may be constituted of, for example, a nucleic acid extracting part, a nucleic acid reaction part, a double-stranded chain-recognizing reacting part, an electrochemical measuring part and a washing part.

In addition, other examples of a probe-immobilized chip based on an electrochemical procedure are disclosed, for example, in the following literature (Hashimoto et al., 1994, Wang et al. 1998). This literature is incorporated herein by reference.

(A) Preparation of Probe-Immobilized Chip to be Detected by Fluorescent Detecting Method

The nucleic acid detecting probe in accordance with the aforementioned present invention is immobilized on a substrate. As a substrate, any substrates which have previously been used, such as a glass substrate, a silicon substrate and the like can be used. As immobilizing means, immobilization can be performed by the methods known per se to a person skilled in the art, such as means using a spotter and the like, means employing general semiconductor techniques, and the like.

(B) Preparation of Probe-Immobilized Chip to Be Detected by Electrochemical Method

The nucleic acid detecting probe in accordance with the aforementioned present invention is immobilized on a substrate, for example, an electrode substrate by covalent attachment, ion binding, physical adsorption and chemical adsorption. An Example of a probe-immobilized chip, which is detected by an electrochemical method is an automatic gene detecting apparatus in Patent No. 2573443 registered on Oct. 24, 1996, being not limiting. The literature is incorporated herein by reference.

According to another aspect of the present invention, there is provided a probe-immobilized chip comprising a substrate, and a nucleic acid detecting probe in accordance with the aforementioned present invention immobilized on the substrate, and such probe-immobilized chip is within the scope of the present invention.

According to one aspect of the present invention, the aforementioned nucleic acid for detecting the endocrine disrupting property of a chemical substance or a part thereof can be used as a nucleic acid detecting probe for detecting the endocrine disrupting property of a chemical substance. Such a nucleic acid detecting probe for detecting the endocrine disrupting property of a chemical substance is within the scope of the present invention.

Examples of a probe-immobilized chip in accordance with aspects of the present invention will be shown below.

### FIRST EXAMPLE

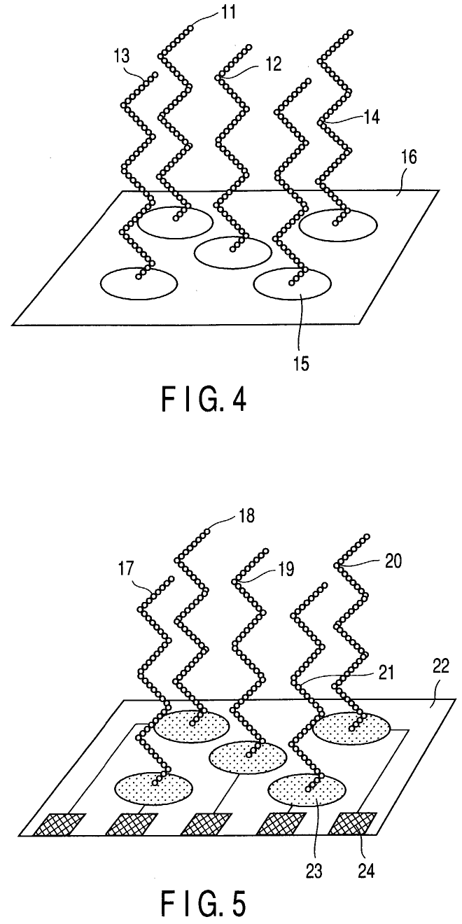

FIG. 4 schematically shows a first Example of a probe-immobilized substrate, probe-immobilized chip, which can be used in accordance with an aspect of the present invention. The probe-immobilized substrate which is the first Example is provided with a substrate 16 and 1 or more probes 11 to 14 immobilized on 1 or more immobilizing regions 15 present on the surface of the substrate (FIG. 4). In accordance with an aspect of the present invention, at least, for example, a first probe as a probe 11 may be immobilized, and a second probe as a probe 12 may be immobilized.

Such probe-immobilized substrate can be prepared, for example, by immobilizing a probe on a substrate such as a silicon substrate by the means known per se.

In accordance with an aspect of the present invention, the number of immobilizing regions 15 to be arranged on one substrate, and the number of probes to be immobilized thereon are not limited to specified ones, and may be changed if necessary. Alternatively, a plurality kinds of nucleotide sequences as a probe may be arranged on one substrate. An immobilizing pattern by which a plurality of and/or a plurality kinds of probes are immobilized on a substrate can be appropriately designed and changed by a person skilled in the art, if necessary. Such probe-immobilized substrates are within the scope of the present invention.

### SECOND EXAMPLE

A second Example of a probe-immobilized substrate which can be used in an aspect of the present invention will be explained by using FIG. 5. A probe-immobilized substrate which is a second Example is provided with probes 17 to 21 immobilized on 1 or more electrodes 23 disposed on a substrate 22 (FIG. 5). An electrode 23 is connected to a pad 24 from which electrical information is taken out.

Such probe-immobilized substrate can be prepared, for example, by arranging an electrode on a substrate such as a silicon substrate and immobilizing a probe on the surface of an electrode by the means known per se,. In accordance with an aspect of the present invention, at least, for example, a first probe as a probe 17 may be immobilized, and a second probe as a probe 18 may be immobilized.

In the present aspect, the number of electrodes is 5, but the number of electrodes to be arranged on one substrate is not limited to this. In addition, a pattern of arranging electrodes is not limited to that shown in FIG. 5, but can be appropriately designed and changed by a person skilled in the art. If necessary, a reference electrode and a counter electrode may be disposed. Such probe-immobilized substrate is within the scope of the present invention.

In the case of the probe-immobilized substrates for detecting fluorescence as described in the above examples, a probe may be immobilized on any of substrates. Alternatively, in the case of a probe-immobilized substrate for performing electrochemical detection as described in the second example, an electrode is arranged on any of the substrates so that electrochemical detection is possible, and a probe may be immobilized on an electrode.

V. Nucleic Acid Detecting Primer

According to one aspect of the present invention, the aforementioned nucleic acid for detecting the endocrine disrupting property of a chemical substance or a part thereof can be also used as a nucleic acid detecting primer for detecting the endocrine disrupting property of a chemical substance. Such nucleic acid detecting primer for detecting the endocrine disrupting property of a chemical substance is within the scope of the present invention.

According to the present invention, a preferable nucleic acid detecting primer for detecting the endocrine disrupting property of a chemical substance may be a nucleic acid detecting primer containing a nucleic acid comprising consecutive 15 bases to 30 bases contained in a nucleotide sequence selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its complementary sequence, a nucleotide sequence described in SEQ ID No. 5 and its complementary sequence, a nucleotide sequence described in SEQ ID No. 14 and its complementary sequence, a nucleotide sequence described in SEQ ID No. 15 and its complementary sequence, a nucleotide sequence described in SEQ ID No. 23 and its complementary sequence, a nucleotide sequence described in SEQ ID No. 27 and its complementary sequence, and a nucleotide sequence described in SEQ ID No. 28 and its complementary sequence.

In addition, a nucleic acid detecting primer for detecting the endocrine disrupting property of a chemical substance may contain a nucleic acid comprising consecutive 15 bases to 30 bases contained in a nucleotide sequence selected from a group consisting of a nucleic acid sequence having 85% to 100% homology with a nucleotide sequence described in SEQ ID No. 1 and its complementary sequence, a nucleic acid sequence having 85% to 100% homology with a nucleotide sequence described in SEQ ID No. 3 and its complementary sequence, a nucleic acid sequence having 85% to 100% homology with a nucleotide sequence described in SEQ ID No. 4 and its complementary sequence, a nucleic acid sequence having 85% to 100% homology with a nucleotide sequence described in SEQ ID No. 5 and its complementary sequence, a nucleic acid sequence having 85% to 100% homology with a nucleotide sequence described in SEQ ID No. 14 and its complementary sequence, a nucleic acid sequence having 85% to 100% homology with a nucleotide sequence described in SEQ ID No. 15 and its complementary sequence, a nucleic acid sequence having 85% to 100% homology with a nucleotide sequence described in SEQ ID No. 23 and its complementary sequence, a nucleic acid sequence having 85% to 100% homology with a nucleotide sequence described in SEQ ID No. 27 and its complementary sequence, and a nucleic acid sequence having 85% to 100% homology with a nucleotide sequence described in SEQ ID No. 28 and its complementary sequence.

Still further, preferable examples of a nucleic acid detecting primer for detecting the endocrine disrupting property of a chemical substance include, for the purpose of detecting ND83-3, nucleic acids shown by nucleotide sequences described in SEQ ID No. 6, SEQ ID No. 7, SEQ ID No. 10, and SEQ ID No. 11. More preferably, SEQ ID No. 6 and SEQ ID No. 7, SEQ ID No. 10 and SEQ ID No. 11 are used as a forward primer and a reverse primer, respectively.

For detecting ND83-4, preferably, nucleic acids shown by nucleotide sequences described in SEQ ID No. 8, SEQ ID No. 9, SEQ ID No. 12 and SEQ ID No. 13 are included. In addition, more preferably, SEQ ID No. 8 and SEQ ID No. 9, SEQ ID No. 12 and SEQ ID No. 13 are used as a forward primer and a reverse primer, respectively.

For detecting ND118-1, preferably, nucleic acids shown by nucleotide sequences described in SEQ ID No. 17 and SEQ ID No. 18 are included. More preferably SEQ ID No. 17 and SEQ ID No. 18 are used as a forward primer and a reverse primer, respectively.

For detecting ND818-2, preferably, nucleic acids shown by nucleotide sequences described in SEQ ID No. 19, SEQ ID No. 20, SEQ ID No. 21 and SEQ ID No. 22 are included. In addition, more preferably, SEQ ID No. 19 and SEQ ID No. 20, SEQ ID No. 21 and SEQ ID No. 22 are used as a forward primer and a reverse primer, respectively.

For detecting ND87-3, preferably, nucleic acids shown by nucleotide sequences described in SEQ ID No. 25 and SEQ ID No. 26 are included. More preferably, SEQ ID No. 25 and SEQ ID No. 26 are used as a forward primer and reverse primer, respectively.

In accordance with one aspect of the present invention, a nucleic acid detecting primer for detecting the endocrine disrupting property of a chemical substance may contain a desired labeling substance. In addition, a nucleic acid detecting primer for detecting the endocrine disrupting property of a chemical substance may contain a further sequence in addition to the aforementioned nucleic acid.

VI. Method for Detecting Endocrine Disrupting Property Using Nucleic Acid Detecting Primer.

By using such a nucleic acid detecting primer, it is possible to detect the endocrine disrupting property of a chemical substance by the following procedures. First, a chemical substance which is a test subject is made to act on a specimen. Then, a specimen nucleic acid is prepared from the above specimen. The resulting specimen nucleic acid is amplified using the primer in accordance with the aforementioned present invention and a nucleic acid amplifying enzyme. Then, according to the same conditions except that the above chemical substance is not acted, a control nucleic acid taken from a treated specimen and the resulting control amplified product may be compared.

As used herein, the term “specimen” refers to the living substance on which a chemical substance which is a test subject is acted. Examples of the specimen may be organism individuals such as mouse, rat, cat, dog, cow, goat, pig, sheep and monkey, or may be cultured cells and tissues derived from animal living body including human being.

In addition, when organism individuals are used as a specimen, a specimen nucleic acid may be prepared from blood, serum, lymph liquid and tissue obtained from the individual. In this case, if needed, necessary arbitrary pretreatment such as homogenization and extraction may be performed, and such pretreatment can be selected by a person skilled in the art depending on a sample which is to be a subject. In addition, when a specimen is cultured cell or tissue, the following extraction of a specimen nucleic acid may be performed after the similar pretreatment is performed, or extraction may be performed without pretreatment.

A specimen nucleic acid may be prepared from a used specimen by the means known per se. As used herein, the term “specimen nucleic acid” generally refers to a mRNA or whole RNA which is expressed in a subject. A step of obtaining a specimen nucleic acid from a subject can be performed by the means known per se. For example, a commercially available kit may be employed, or a solid-liquid extraction method using a carrier such as an oligo dT column may be employed, for example, a liquid-liquid extraction method such as phenol-chloroform and the like may be performed, being not limited to them.

A nucleic acid amplifying enzyme which can be used herein may be any enzymes to be used for amplifying a nucleic acid. For example, the enzyme may be a DNA polymerase or a DNA polymerase having a reverse transcription activity.

VII. Peptide Whose Expressed Amount is Specifically Influenced by Endocrine Disrupting Property

According to one aspect of the present invention, there is provided a peptide derived form a gene whose expressed amount in a specimen is specifically influenced by the endocrine disrupting property. Such peptide can be also utilized for detecting the endocrine disrupting property of a chemical substance.

As used herein, the term “peptide” refers to a substance which is constructed of plural amino acids by binding each other by peptide bond, and comprehensively refers to peptide consisting of some amino acids linked by peptide bond, polypeptide consisting of many amino acid linked by peptide bond, as well as protein consisting of simplex or plural kinds of polypeptides.

According to the present invention, a peptide for detecting the endocrine disrupting property of a chemical substance may be a peptide encoded by a nucleic acid represented by a nucleotide sequence selected from a group consisting of a nucleotide sequence described in SEQ ID No. 1, a nucleotide sequence described in SEQ ID No. 3, a nucleotide sequence described in SEQ ID No. 15, and a nucleotide sequence described in SEQ ID No. 23. For example, such peptide may be a peptide described in SEQ ID No. 2, SEQ ID No. 16 or SEQ ID No. 24.

Further, it may be a peptide encoded by a nucleic acid represented by a nucleotide sequence selected from a group consisting of a nucleic acid sequence having 85%-100% homology with a nucleotide sequence described in SEQ ID No. 1, a nucleic acid sequence having 85% to 100% homology with a nucleotide sequence described in SEQ ID No. 3, a nucleic acid sequence having 85% to 100% homology with a nucleotide sequence described in SEQ ID No. 15, and a nucleic acid sequence having 85% to 100% homology with a nucleotide sequence described in SEQ ID No. 23.

Moreover, a peptide for detecting the endocrine disrupting property of a chemical substance may be a protein containing the aforementioned peptide.

(1) Preparation of Peptide Encoded by ND83-3

A peptide for detecting the endocrine disrupting property of a chemical substance in accordance with one aspect of the present invention can be prepared, for example, as follows:

A peptide encoded by ND83-3 can be obtained using a nucleic acid sequence provided by SEQ ID No. 1 employing a recombinant DNA technique. For example, by recombining a coding region of a peptide contained in SEQ ID No. 1 into a known expression vector such as pGEX, pET and pYES, a peptide encoded by ND83-3 can be prepared in a large amount in a prokaryote such as Escherichia coli, and an eukaryote such as yeast, an insect cell, a mammal cell and a plant cell. The peptide obtained at this time may be a fused peptide with other protein depending on the system of the expression vector.

Alternatively, a peptide may be prepared by incorporating a nucleic acid sequence provided by SEQ ID No. 1 into a vector such as pBlueScriptII, preparing a RNA by in vitro transcription, and performing in vitro translation using this as a template. Alternatively, a peptide can be synthesized by chemical synthesis based on an amino acid sequence provided by SEQ ID No. 1.

In addition, similarly, peptides encoded by ND818-2 and ND87-3 can be prepared, respectively, based on nucleotide sequences represented by SEQ ID No. 15 and SEQ ID No. 23.

Further, desired peptides can be prepared based on SEQ ID Nos. 1, 3, 15 and 23 by the method known per se. Such peptides are within the scope of the present invention.

Amino acid sequences represented by SEQ ID No. 2, SEQ ID No. 16 and SEQ ID No. 24 are an Example of peptides obtained as described above.

In addition, according to another aspect of the present invention, there are provided a polyclonal antibody and a monoclonal antibody which recognize the aforementioned peptides. For example, such polyclonal antibody and monoclonal antibody can be prepared by the method known per se. Examples of methods for preparing a polyclonal antibody and a monoclonal antibody, respectively, are shown below.

(2) Preparation of Polyclonal Antibody Recognizing Peptide

The peptide obtained by the method described in the aforementioned (1) “Preparation of peptide encoded by ND83-3” as an antigen is administered to, for example, rabbit, goat, rat, mouse or hamster. As a peptide to be administered as an antigen, a peptide which is covalently bound to a carrier protein such as bovine thyroglobulin may be used. Administration of an antigen is performed 3 to 10 times every 1 to 2 weeks after first administration, 3 to 7 days after each administration, blood is taken, and it is confirmed by ELISA that serum is reacted with an antigen to be used in immunization. Serum is obtained from an animal whose serum showed a sufficient antibody titer, and separated and purified to obtain a polyclonal antibody. When a peptide used as an antigen is a fused peptide with other protein, for example, it is necessary to remove an antibody recognizing a part other than a peptide part, employing an affinity column on which a protein part fused with a peptide is immobilized.

(3) Preparation of Monoclonal Antibody Recognizing Peptide

A hybridoma group is prepared by fusing a mouse spleen cell immunized with the peptide obtained in the (1) “Preparation of peptide encoded by ND83-3” as an antigen with a myelome cell. The peptide prepared in (1) is immobilized, for example, on a microtiter plate for ELISA, to prepare an assay plate. Only a hybridoma producing an antibody specifically recognizing a peptide is selected from the prepared hybridoma group employing the aforementioned assay plate. When a peptide used as an antigen is a fused peptide with other protein, it is necessary to prepare an assay plate on which only a protein part fused with a peptide is immobilized, and to remove an antibody recognizing the fused peptide. The resulting hybridoma is cloned by, for example, a limiting dilution method and injected into a mouse intraperitoneally to obtain a monoclonal antibody produced by the hybridoma.

According to another aspect of the present invention, there is provided a method of detecting the endocrine disrupting property by utilizing the aforementioned an antibody selected from a group consisting of a polyclonal antibody and a monoclonal antibody, and detecting an antigen which specifically binds thereto.

As used herein, the term “specimen sample” refers to a desired sample taken from a specimen. For example, when a specimen is an individual, a sample may be blood, serum, lymph liquid or tissue obtained from the individual, or may be a cultured cell, a cultured tissue or a culturing solution thereof. Alternatively, if needed, they may be subjected to necessary arbitrary pretreatment such as homogenization and extraction. Such pretreatment can be selected by a person skilled in the art depending on a sample which is to be a subject.

Such a method can be performed as follows. First, a chemical substance is made to act on a specimen and, thereafter, a specimen sample is obtained from the aforementioned specimen. Then, the specimen sample is made to react with at least one detecting probe selected from a group consisting of a polyclonal antibody and a monoclonal antibody which recognize the peptide. After the reaction, the existence of the target substance is detected by detecting the binding of the aforementioned detecting probe and a target substance. By comparing the results obtained by this detection, with results of detection of the existence of the target substance in the case where the compound has not acted on a specimen, it becomes possible to detect the endocrine disrupting property of the chemical substance.

Such detection may be performed by using a probe-immobilized chip (generally, referred to as protein chip) prepared by immobilizing the antibody on a substrate. Thereby, it becomes possible to detect such peptide simply. Such probe-immobilized chip is included within the scope of the present invention. Such probe-immobilized chip can be prepared as follows.

(4) Preparation of Protein Chip

The antibody obtained by the method described in (2) “Preparation of polyclonal antibody recognizing peptide” or (3) “Preparation of monoclonal antibody recognizing peptide” can be integrated on a substrate, for example, on a glass substrate using a commercially available spotter, to prepare a probe-immobilized chip which can detect the endocrine disrupting property of a chemical substance.

### EXAMPLES

### Example 1

### Search of Gene Having High TCDD Responsiveness

(1) Culturing of Neuro2a

First, triiodetyronine-removed bovine fetal serum to be used for culturing mouse neuroblastoma Neuro2a was prepared. Anionic exchange resin AG1-X8 and bovine fetal serum were mixed at a ratio of resin 50 mg per 1 mL of serum, and incubated at room temperature for 5 hours. The resin was removed by centrifugation at 1000×g for 10 minutes. Then, a fresh aforementioned resin was added at a ratio of resin 50 mg per 1 mL of serum, and further incubated at room temperature for 18 hours. Thereafter centrifugations at 1000×g and 30,000×g were performed for 20 minutes, respectively, to remove the resin completely. Then, sterilization was performed by a filter having a pore size of 0.22 μm, to prepare triiodetyronine-removed bovine fetal serum.

DF medium used for culturing Neuro2a was prepared by mixing Dulbecco's MEM medium containing 10% triiodotyronine-removed bovine serum obtained by the aforementioned method and Ham's medium at a ratio of 1:1.

First, Neuro2a was cultured in the DF medium at 37° C. for 1 day in the presence of 5% carbon dioxide. Then, 30 nM triiodotyronine was added to the culturing solution, followed by culturing 37° C. for 3 days in the presence of carbon dioxide. After this culturing, the resulting cells were classified into two conditions by adding into two culturing containers. To one of conditions was added 2,3,7,8-tetrachloro-benzo-p-dioxin (hereinafter, referred to as TCDD) to the final concentration of 10 nM (hereinafter, referred to as TCDD-added condition). On the other hand, to the other condition was added no TCDD (hereinafter, referred to as TCDD-not added condition). Neuro2a's in these two conditions were cultured for 24 hours.

(2) Comparison of Genes Expressed in TCDD-Added Condition and TCDD-Not Added

Genes expressed in cells in two conditions (TCDD-added condition and TCDD-not added condition) cultured by the method described in the above (1) were compared using the differential display method.

First, the whole RNAs were extracted from two conditions described in then above (1), respectively. This extraction was performed using RNA Extraction Kit (manufactured by Pharmacia) according to the annexed manual. The whole RNA obtained by this extraction was treated with DNase I to remove a DNA. Thereafter, an amount of the whole RNA contained in the resulting solution was measured by a spectrophotometer, and the concentration was adjusted so that RNA per 1 μL became 250 pmol.

The fluorescent differential display method was performed using TaKaRa FDD Kit Fluorescein Version 1.1 (manufactured by Takarashuzo) according to the annexed manual.

As a primer for synthesizing a cDNA from the RNA, Downstream Primer No. 1 and No. 8, that is, modified (dT) primer, annexed to the above kit, which was designed as an anchor primer.

For PCR, the same anchor primer as that for synthesis of a cDNA (that is, Downstream Primer No. 1 and No. 8; this is modified (dT) primer) was used as a downstream primer. As an upstream primer, 24 kinds of primers of Upstream Primer No. 1 to No. 24 which are random primers annexed to the kit were used.

Regarding the RNA extracted from the aforementioned (1) TCDD-added condition and TCDD-not added condition, the aforementioned primers were used to synthesize a cDNA and, further, PCR was performed using the aforementioned primers, and electrophoresis was performed with 4% polyacrylamide gel. By this electrophoresis, the PCR amplified products were separated based on molecular weight. After separation, the polynucleotides in the gel were visualized with a fluorescent image scanner. At that status, 10 nM TCDD added condition and TCDD-not added condition were compared, and bands different in the concentration were excised from the gel. Sterilized water was added to the gel containing the bands obtained by excision and allowed to stand for 30 minutes or longer. Thereafter, for this, hot extraction was performed at 100° C. for 10 minutes, to recover a polynucleotide. Further, reamplification was performed under the aforementioned conditions, and purification was performed by electrophoresis using agarose gel. The resulting seven gene fragments were sequenced. The polynucleotides which were the resulting gene fragments were designated ND81-1, ND83-2, ND83-3, ND83-4, ND118-1, ND818-2 and ND87-3, respectively.

(3) Sequences of ND83-3, ND83-4, ND118-1, ND813-2 and ND87-3

Nucleotide sequences of ND83-3, ND83-4, ND118-1, ND818-2 and ND87-3 which were sequenced in the above (2) are shown in SEQ ID No. 3, SEQ ID No. 5, SEQ ID No. 14, SEQ ID No. 27 and SEQ ID No. 28. As an Example of a PCR primer which can specifically amplify these, the following primers were designed:

As a PCR primer for amplifying ND83-3, an upstream primer is a polynucleotide having a nucleotide sequence described in SEQ ID No. 6, and a downstream primer is a polynucleotide having a nucleotide sequence described in SEQ ID No. 7.

As a PCR primer for amplifying ND83-4, an upstream primer is a polynucleotide having a nucleotide sequence described in SEQ ID No. 8, and a downstream primer is a polynucleotide having a nucleotide sequence described in SEQ ID No. 9.

As a PCR primer for amplifying ND118-1, an upstream primer is a polynucleotide having a nucleotide sequence described in SEQ ID No. 17, and a downstream primer is a polynucleotide having a nucleotide sequence described in SEQ ID No. 18.

As a PCR primer for amplifying ND818-2, an upstream primer is a polynucleotide having a nucleotide sequence described in SEQ ID No. 19, and a downstream primer is a polynucleotide having a nucleotide sequence described in SEQ ID No. 20.

As a PCR primer for amplifying ND87-3, an upstream primer is a polynucleotide having a nucleotide sequence described in SEQ ID No. 25, and a downstream primer is a polynucleotide having a nucleotide sequence described in SEQ ID No. 26.

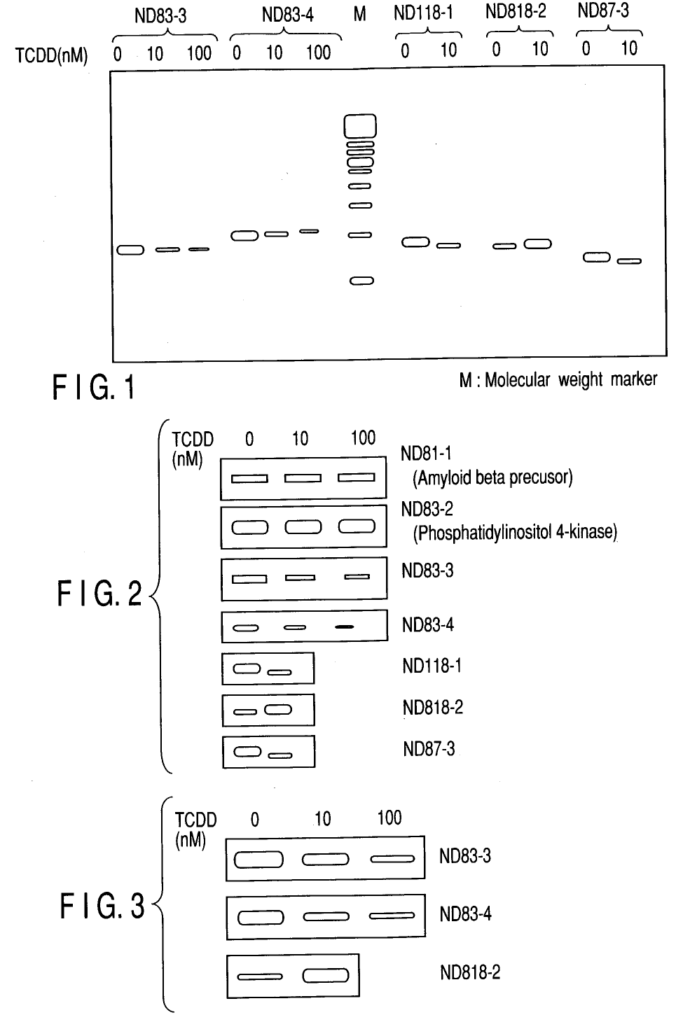

Freshly, Neuro2a was cultured under the same conditions as those described in the above (1) except that the concentration of added TCDD was 0, 10 and 100 nM. In those respective conditions, the whole RNA was extracted. After a reverse transcription reaction from the extracted whole RNA was performed using a (dT) primer, a PCR was conducted. In this PCR, the aforementioned ND83-3, ND83-4, ND118-1, ND818-2 and ND87-3 were amplified using PCR primers which are specific for them, respectively. The results are shown in FIG. 1.

As apparent from FIG. 1, ND83-3, ND83-4, ND118-1, ND818-2 and ND87-3 were specifically amplified by the PCR. Expressed amounts of ND83-3 and ND83-4 were reduced by addition of 10 nM TCDD (FIG. 1). When 100 nM was added, expressed amounts were further reduced as compared with addition of 10 nM (FIG. 1). Therefore, it was confirmed that ND83-3 and ND83-4 are responsive to TCDD. In addition, expressed amounts of ND118-1 and ND87-3 were reduced by addition of 10 nM TCDD (FIG. 1). An expressed amount of ND818-2 was increased by addition of 10 nM TCDD (FIG. 1). Therefore, it was confirmed that ND118-1, ND818-2 and ND87-3 are responsive to TCDD.

According to an aspect of the present invention, a TCDD-responsive gene is provided. In addition, based on a nucleic acid sequence of the gene, a nucleic acid detecting primer for specifically detecting the endocrine disrupting activity of a chemical substance is provided.

(4) Selection of TCDD-Responsive Gene

Regarding a group of gene fragments obtained in the above (2), TCDD responsivenesses were compared.

First, based on nucleotide sequences obtained in the above (2), PCR primers which can specifically amplify respective gene fragments were designed.

On the other hand, Neuro2a was cultured according to the same conditions as those of the above (1) except that the concentration of added TCDD was 0, 10 and 100 nM. In those respective conditions, the RNA was extracted. A reverse transcription reaction from the extracted whole RNA was performed using a (dT) primer. Thereafter, respective gene fragments were amplified using PCR primers which can specifically amplify respective gene fragments. The amplified products were subjected to electrophoresis, and the resulting bands were compared to expressed amounts by TCDD.

Regarding seven of gene fragments which showed responsiveness to TCDD, the results are shown in FIG. 2. As shown in FIG. 2, expressed amounts of 5 genes of ND83-3, ND83-4, ND118-1, ND818-2 and ND87-3 were remarkably changed by addition of TCDD. Therefore, since ND.83-3, ND83-4, ND118-1, ND818-2 and ND87-3 are gene fragments having the high TCDD responsiveness, they were selected as a gene marker which can be used for determination of the endocrine disrupting property of a chemical substance.

In accordance with an aspect of the present invention, a nucleic acid detecting primer for specifically detecting the endocrine disrupting activity of a chemical substance was provided.

### Example 2

### Identification of ND83-3, ND83-4, ND118-1ND818-2 and ND87-3

Of gene fragments whose nucleotide sequences were determined in Example 1, homology search was performed regarding ND83-3 and ND83-4. Homology search was performed by executing the FASTA program on the database of the Japan DNA Data Bank (DDBJ). As a result, only IMAGE:3496023 showed 85% or more homology with ND83-3. The homology was 94%. In addition, this clone was the EST (Expressed Sequence Tags) clone registered by the NIH (National Institute of Health), and the function of the gene was unknown. A nucleotide sequence of a full length cDNA of ND83-3 revealed by this identification is described in SEQ ID No. 1. Further, one Example of a sequence of an amino acid encoded by this cDNA is described in SEQ ID No. 2.

In addition, there were 6 clones which show 85% or more homology with ND83-4. RIKEN:G431004A07 is a mouse EST clone registered by The Institute of Physical and Chemical Research (RIKEN, Saitama, Japan), and the function of the gene was unknown. IMAGE:3156947, IMAGE:540238, IMAGE:805651 and UI-M-CD1-azs-c-11-0-UI are all mouse EST clones registered by NIH, and the functions of genes are all unknown. Moreover, mouse RP24-388P13 is a mouse genomic clone, and the function of the gene was unknown. A nucleotide sequence of a full length cDNA of ND83-4 revealed by this identification is described in SEQ ID No. 4.

Homology search was also performed regarding ND118-1, ND818-2 and ND87-3. Homology search was performed by executing the SSAHA program on the database for a mouse genome. 252 base pairs which are corresponding to an almost full length of ND118-1 showed 100% homology with a region of from 107213067 to 107213304 on mouse 11^{th} chromosome, but information regarding a gene, the function of which is known, was not obtained from the same region.

ND818-2 was encoded in a region of from 105476953 to 1054477260 on mouse 3^{rd} chromosome. In the neighborhood of the same region was encoded mouse hypothetical protein MGC:7720 (Accession No. NM030249) which is a function-unknown gene within a reference sequence (Refseq) registered by NCBI. A nucleotide sequence of a full length cDNA of ND818-2 revealed by this identification is described in SEQ ID No. 15. Further, one Example of a sequence of an amino acid encoded by this cDNA is described in SEQ ID No. 16.

ND87-3 was encoded in a region of from 7646283 to 7646436 on mouse 10^{th} chromosome. In the same region was encoded a function-unknown gene (Accession No. AK014406) predicted by Ensembl. A nucleotide sequence of a full length cDNA of ND87-3 revealed by this identification is described in SEQ ID No. 23. Further, one Example of a sequence of an amino acid encoded by this cDNA is described in SEQ ID No. 24.

### Example 3

### Further Primer

Based on nucleotide sequences of ND83-3, ND83-4 and ND818-2 determined in the above Example 2, further primers were designed.

Further primers which are specific for ND83-3 are a forward primer having a nucleotide sequence described in SEQ ID No. 10 and a reverse primer having a nucleotide sequence described in SEQ ID No. 11.

Further primers which are specific for ND83-4 are a forward primer having a nucleotide sequence described in SEQ ID No. 12 and a reverse primer having a nucleotide sequence described in SEQ ID No. 13.

Further primers which are specific for ND818-2 are a forward primer having a nucleotide sequence described in SEQ ID No. 21 and a reverse primer having a nucleotide sequence described in SEQ ID No. 22.

The whole RNA was extracted from cells cultured under two conditions (10 nM TCDD-added condition and TCDD-not added condition) cultured by the method described in the aforementioned Example 1 (1). Regarding the resulting extract products, a reverse transcription PCR method was performed using the aforementioned primers, and expressed amounts of respective genes were compared. As a result, expressed amounts of ND83-3 and ND83-4 were reduced by addition of 10 nM of TCDD (FIG. 3). An expressed amount of ND818-2 was increased by addition of 10 nM TCDD (FIG. 3).

According to an aspect of the present invention, a further nucleic acid detecting primer for specifically detecting the endocrine disrupting activity of a chemical substance was provided.

### Example 4

Neuro2a was cultured under the conditions described in the above Example 1, and the whole RNA was extracted regarding 10 nM TCDD-added condition and not added condition. The extracted RNA was subjected to gel electrophoresis under the denaturing conditions, and separation by molecular weight was performed. Then, the RNA after separation was transferred from the aforementioned gel to a nylon membrane.

As nucleic acid detecting probes which can detect nucleic acids ND83-3 and ND83-4 isolated in the aforementioned Example 1, as well as ND118-1, ND818-2 and ND87-3, nucleic acid detecting probes having nucleotide sequences described in SEQ ID No. 11 and SEQ ID No. 13, as well as SEQ ID No. 18, SEQ ID No. 20, SEQ ID No. 22 and SEQ ID No. 26 were used, respectively. First, these nucleic acid detecting probes were chemically labeled. Then, each of them was hybridized with the aforementioned nylon membrane. Thereafter, chemiluminescence was detected.

As a result, when nucleic acid detecting probes corresponding to any of ND83-3 and ND83-4, as well as ND118-1 and ND87-3 were used, a signal obtained by hybridization was reduced by addition of 10 nM TCDD. And when a nucleic acid detecting probe corresponding to ND818-2, a signal obtained by hybridization was increased by addition of 10 nM TCDD.

According to an aspect of the present invention, a method which can specifically detect the endocrine disrupting property by detection using chemiluminescence as an index was provided.

### Example 5

As nucleic acid detecting probes which can detect nucleic acids ND83-3 and ND83-4 isolated in the aforementioned Example 1, as well as ND118-1, ND818-2 and ND87-3, nucleic acid detecting probes having nucleotide sequences described in SEQ ID No. 11 and SEQ ID No. 13, as well as SEQ ID No. 18, SEQ ID No. 20 and SEQ ID No. 26 were used, respectively. These nucleic acid detecting probes were immobilized on substrates to make probe-immobilized chips. Neuro2a was cultured under the conditions described in the above Example 1, the whole RNA was extracted regarding 10 nM TCDD-added condition and not added condition, and fluorescence-labeled. Thereafter, fluorescence-labeled whole RNA was hybridized with a polynucleotide of the aforementioned nucleic acid detecting chip (that is, probe-immobilized chip). As a result, even when nucleic acid detecting probes corresponding to any of ND83-3 and ND83-4, as well as ND118-1 and ND87-3 were used, a signal obtained on the probe-immobilized chip was reduced by addition of 10 nM TCDD. And when a nucleic acid detecting probe corresponding to ND818-2, a signal obtained on the probe-immobilized chip was increased by addition of 10 nM TCDD.

According to an aspect of the present invention, a probe-immobilized chip which can specifically detect the endocrine disrupting property was provided.

### Example 6

As nucleic acid detecting probes which can detect nucleic acids ND83-3 and ND83-4 isolated in the aforementioned Example 1, as well as ND118-1, ND818-2 and ND87-3, nucleic acid detecting probes having nucleotide sequences described in SEQ ID No. 11 and SEQ ID No. 13, as well as SEQ ID No. 18, SEQ ID No. 20 and SEQ ID No. 26 were used, respectively. These nucleic acid detecting probes were immobilized on prescribed positions on different gold electrodes, respectively, to make probe-immobilized chips. Neuro2a was cultured under the conditions described in the aforementioned Example 1, and the whole RNA was extracted regarding 10 nM TCDD-added condition and not added condition. It was reacted with the aforementioned nucleic acid detecting chip. As a reagent for obtaining an electric signal from a hybridized nucleic acid, Hoechst 33258 was used. As a result, current values obtained from any nucleic acid detecting probes corresponding to ND83-3 and ND83-4, as well as ND118-1 and ND87-3 were reduced by addition of 10 nM TCDD. And current values obtained from a nucleic acid detecting probe corresponding to ND818-2 was increased by addition of 10 nM TCDD.

According to an aspect of the present invention, there was provided a probe-immobilized chip, by which detection is conducted electrochemically, being capable of detecting specifically the endocrine disrupting property.

### Example 7

### Preparation of GST Fused Peptides by Escherichia coli

Regarding ND83-3, a GST fused peptide was made as follows: Using an upstream primer with a recognition sequence for restriction enzyme EcoRI added at a 5′-terminal (that is, 5′-GGGAATTCGGACGCGTGGGCTTGATGC-3′) and a downstream primer with a recognition sequence for NotI added at a 5′-terminal (that is, 5′-CCGCGGCCGCTCAAACACTGTGGATGT-3′), a nucleic acid sequence shown in SEQ ID No. 1 was amplified by PCR, which was inserted into EcoRI and NotI sites of an expression vector: pGEX-6P-2 (manufactured by Amersham Bioscience). After a nucleotide sequence was analyzed and it was confirmed that a nucleic acid fragment was correctly inserted in an expression vector, host Escherichia coli BL21 was transformed. After cultured in LB medium at 37° C. for 5 hours, IPTG was added to the final concentration of 0.4 mM, followed by further culturing at 37° C. for 2.5 hours. Thereafter, cells were recovered by centrifugation. The recovered cells were suspended in a lysis solution (50 mM Tris-HCl (pH 7.5), 1 mM EDTA, 1% Tirton X-100, 0.2% SDS, 0.2 mM PMSF) and ground by ultrasound. After grinding, ground cells were centrifuged at 1000 g for 30 minutes, Glutathione Sepharose 4B was added to the supernatant, followed by incubation at 4° C. for 1 hour. After the beads were washed well, a fused peptide was eluted with an eluting solution (10 mM Tris-HCl (pH 7.5), 50 mM glutathione).

In addition, regarding ND818-2, a GST fused peptide was prepared according to the same manner as that for the aforementioned ND83-3 except that an upstream primer with a recognition sequence for restriction enzyme EcoRI added at a 5′-terminal (that is, 5′-CCGGAATTCATGAATCTGGAAAAACTCAGCAAGC-3′) and a downstream primer with a recognition sequence for NotI added at a 5′-terminal (that is, 5′-GGGCGGCCGCTACTGCTGGTAGGCAAAAGTATCTC-3′) were used, and pGEX-6P-1 was used as an expression vector.

Regarding ND87-3, a GST fused peptide was made in the same manner as that for the aforementioned ND83-3 except that an upstream primer with a recognition sequence for restriction enzyme EcoRI added at a 5′-terminal (that is, 5′-CCGGATTCCATGGCGGTTCTCTTGGAGACCACTC-3′) and a downstream primer with a recognition sequence for NotI added at a 5′-terminal (that is, 5′-GGGCGGCCGCTCATCTGTACTTGGATTTTTCTT-3′) were used, and pGEX-6P-3 was used as an expression vector.

### Example 8

### Preparation of Polyclonal Antibody

Using a GST fused peptide prepared in Example 7 as an antigen, 100 μg per rabbit was injected intraperitoneally. After first administration, administration of an antigen was continued 3 to 10 times about every 2 weeks. After administration, blood was taken from eyegrounds vein plexus at 3 to 7 days, and it was confirmed by ELISA that the resulting serum reacts with an antigen used for immunization. Serum was taken from a rabbit showing a sufficient antibody titer against an antigen used for immunization, a GST antibody was removed from a 40% saturated ammonium sulfate precipitation fraction by a GST affinity column, a passing-through fraction was further purified by an antigen column of a GST-derived peptide.

### Example 9

### Preparation of Monoclonal Antibody

(1) Preparation of Hybridoma

The GST fused peptide prepared in Example 7 and a complete adjuvant were taken into a 1 mL syringe, respectively, at an equivalent amount, which were connected by a joint and stirred well to emulsification. This was injected into a mouse intraperitoneally for immunization. Two weeks after first immunization, second immunization was conducted and, further two weeks after, immunized with a booster and, 3 days after booster, cell fusion operations were performed. Upon cell fusion, vertebrae cervicales of immunized mouse was dislocated, spleen was removed, and transferred into a 6 cm dish into which 5 mL of RPMI has been placed. Excess fat was removed from this spleen, and further washed freshly using two 6 cm dishes into which 5 mL of RPMI had been placed. Washed spleen was rubbed into between 5 cm square stainless metal nets which had been doubly folded, and washed two times with 10 mL of PRMI by centrifugation. 0.17M NH₄Cl was added to precipitated cells, and soaked in ice for 5 minutes to hemolyze. After this hemolysis operation, 5 mL of RPMI was added, washed by centrifugation at 1,600 rpm for 5 minutes, and further measured up to 20 mL to obtain a spleen cell suspension. On the other hand, 2 to 4 dishes of myeloma cells at logarithmic growing phase were collected by centrifugation at 1,200 rpm for 5 minutes, washed with serum-free RPMI two times, and finally suspended in 10 mL of RPMI to obtain a myeloma cell suspension. The number of cells of the spleen cell suspension and the myeloma cell suspension were counted, and the myeloma cell suspension was added to the spleen cell suspension so that the number of myeloma cells became ⅕ to 1/20 the number of the spleen cells. This myeloma cell suspension was centrifuged for 5 minutes to remove the supernatant, 0.3 mL of 50% polyethylene glycol 1500 was added to the resulting precipitate at once, and immediately stirred well. While continuing to stir, 40 mL of RPMI was added. This suspension was centrifuged at 1,000 rpm for 5 minutes, 50 mL of HAT medium (RPMI-FCS containing the final concentration of 1×10⁻⁷M hypoxanthine, 4×10⁻⁴M aminopterin, 16×10⁻⁴M thymidine) was added to the resulting precipitate, and 100 μL/well was seeded on about three sheets of 96-well plate. 4 to 5 days after, about 100 μL/well of HAT medium was added to the 96-well plate. As a result, hybridoma was grown in almost all wells in about 1 week.

(2) Preparation of Assay Plate in Which Peptide is Solid Phased

The GST fused peptide made in Example 7 was dissolved in PBS to 30 μg/mL, and 50 μL portions were dispensed in a 96-well microtiter plate for ELISA. After allowed to stand at 4° C. overnight, the solution was suction-removed to obtain an assay plate for selecting a hybridoma. By the similar method, an assay plate on which only a GST protein was adsorbed was also made.

(3) Selection of Hybridoma Which Produces Antibody Recognizing Peptide

The assay plate for a GST fused protein made in the above (2) was blocking-treated with 1% BSA-PBS for 1 hour, 20 μL of 2-fold diluted culture supernatant of a hybridome prepared in the above (1) was added thereto, and allowed to stand at 4° C. for 16 hours. The assay plate was washed with 1% BSA-PBS three times to remove an unbound antibody, 50 μL of a solution of protein A fluorescently-labeled with fluorescein was added to a well, allowed to stand at room temperature for 2 hours, and washed with 1% BSA-PBS to remove an unbound protein A. After washing, the assay plate was subjected to a fluorescence detector, and a hybridome present in a well from which fluorescein fluorescence was observed was removed as a hybridoma which produces an antibody recognizing a GST protein. Then, the same operations as those described above were performed using an assay plate for a GST fused protein, and a hybridome present in a well from which fluorescein fluorescence was observed was selected as a hybridoma which produces an antibody recognizing a peptide specifically.

(4) Cloning

The hybridoma selected in the above (3) was taken out, a part thereof was used to count the number of cells with a hemocytometer. The hybridoma was appropriately diluted, and 40 hybridomas and about 1×10⁸ spleen cells prepared from a mouse were mixed in 40 mL RPMI. 200 μL portions of a mixed cell suspension were added onto about two sheets of 96-well plates, and cultured at 37° C. The medium was exchanged two times per week, and culturing was continued for about two weeks. After about two weeks, the supernatant of a well was taken, and the antibody activity was confirmed. A hybridoma in a well exhibiting the antibody activity was further cultured on a 24-well plate. After the cell density was sufficiently heightened by this culturing, the hybridoma was cultured on a 35 mm plate. After similar operations were repeated once more, the hybridoma was frozen and stored as an established hybridoma.

(5) Selection of Monoclonal Antibody Recognizing Peptide

0.5 mL of pristane was injected into an about 4 weeks aged mouse intraperitoneally. About 1 week after pristane injection, hybridomas which had been cultured in advance were collected by centrifugation, and dispersed to about 4×10⁶/mL with RPMI which had been warmed to 37° C. in advance. Of it, 500 μL was injected into a mouse intraperitoneally. About two weeks after, at a stage when ascites was pooled in an abdomen of a mouse and the abdomen was swollen, the abdomen of a mouse was incised, and ascites was sampled using a Pasteur pipette. 0.2 mL of sampled ascites was placed into a 15 mL centrifuge tube containing a NaN₃EDTA solution. After centrifugation at 2,000 rpm for 10 minutes, the resulting supernatant was obtained as a monoclonal antibody solution.

### Example 10

### Detection of Endocrine Disrupting Property of Chemical Substance Using Antibody Against Peptide

A protein was extracted from cells on which TCDD was acted by the method described in Example 1 (1), and subjected to SDS polyacrylamide gel electrophoresis. After run, the gel was detached from a glass plate, placed into a plastic container containing distilled water to wash, and the protein in the gel was transferred onto a PVDF membrane by an electrotransfer method. The PVDF membrane was soaked in Blockace (Yukijirushinyugyo) to perform blocking at room temperature for 2 hours. Subsequently, this was soaked in the antibody obtained in Example 9 and diluted with PBS, to react with the antibody at room temperature for 2 hours. After 2 hours, the PVDF membrane was transferred to TBS to wash for 15 minutes three times, and soaked into 10% Blockace containing secondary anti-mouse-rabbit antibody labeled with peroxidase to react at room temperature for 1 hour. After completion of a reaction with the secondary antibody, the PVDF membrane was soaked in a developing solution (50 mg/mL DAB, 50 mM Tris-HCl buffer (pH 7.5), hydrogen peroxide) to develop color.

### Example 11

### Preparation of Fluorescence-Detecting Type Protein Chip

A glass plate coated with biotinated BSA was further coated with streptoavidin. The antibody obtained in Example 10 which recognizes the peptide was biotynated, and immobilized on a glass substrate using a commercially available spotter, to make a fluorescence-detecting type protein chip.

### Example 12

### Preparation of Current-Detecting Type Protein Chip

The antibody obtained in Example 9 which recognizes the peptide was spotted on a gold electrode with a commercially available spotter to chemically connect thereto, to make a current-detecting type protein chip.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A nucleic acid to detect an endocrine disrupting property of a chemical substance, wherein a nucleotide sequence of the nucleic acid is selected from a group consisting of a nucleotide sequence described in SEQ ID No. 1 and its complementary sequence, a nucleotide sequence described in SEQ ID No. 3 and its complementary sequence, a nucleotide sequence described in SEQ ID No. 4 and its complementary sequence, a nucleotide sequence described in SEQ ID No. 5 and its complementary sequence, a nucleotide sequence described in SEQ ID No. 14 and its complementary sequence, a nucleotide sequence described in SEQ ID No. 15 and its complementary sequence, a nucleotide sequence described in SEQ ID No. 23 and its complementary sequence, and a nucleotide sequence described in SEQ ID No. 27 and its complementary sequence, and a nucleotide sequence described in SEQ ID No. 28 and its complementary sequence.

2. A nucleic acid having 85% to 100% homology with a nucleotide sequence as defined in claim 1.

3. A nucleic acid comprising consecutive 15 based to 30 bases contained in a nucleotide sequence as defined in claim 1.

4. A nucleic acid detecting probe comprising a nucleic acid as defined in claim 3.

5. A nucleic acid detecting probe comprising a nucleotide sequence selected from a group consisting of nucleotide sequences described in SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 17, SEQ ID No. 18, SEQ ID No. 19, SEQ ID No. 20, SEQ ID No. 21, SEQ ID No. 22, SEQ ID No. 25 and SEQ ID No. 26.

6. A nucleic acid detecting primer comprising a nucleotide sequence selected from a group consisting of nucleic sequences described in SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 17, SEQ ID No. 18, SEQ ID No. 19, SEQ ID No. 20, SEQ ID No. 21, SEQ ID No. 22, SEQ ID No. 25 and SEQ ID No. 26.

7. A probe-immobilized chip comprising a substrate, and at least one kind of a nucleic acid detecting probe as defined in claim 4, the nucleic acid detecting probe being solid-phased on the substrate.

8. A probe-immobilized chip according to claim wherein the substrate includes an electrode, the nucleic acid detecting probe is immobilized on the electrode, and hybridization of a target nucleic acid with the nucleic acid detecting probe is electrochemically detected.

9. A method of detecting an endocrine disrupting property of a chemical substance, comprising:
(a) making the chemical substance act on a specimen;
(b) after the chemical substance has acted, obtaining a specimen nucleic acid from the specimen;
(c) making the specimen nucleic acid react with the nucleic acid detecting probe as defined in claim 4;
(d) obtaining an extent of expression of the target nucleic acid by detecting hybridization between a nucleic acid detecting probe and a target nucleic acid; and
(e) detecting the endocrine disrupting property of the chemical substance by comparing an extent of expression of the target nucleic acid obtained in (d) with an extent of expression of the target nucleic acid when the compound has not acted on a specimen.

10. A method of detecting the endocrine disrupting property of a chemical substance according to claim 9, wherein the specimen nucleic acid is a mRNA and, prior to (c), a specimen nucleic acid obtained in (b) is reverse-transcribed using a (dT) primer and a reverse transcriptase, and the resulting reverse transcription product is amplified to obtain the amplified product.

11. A method of detecting the endocrine disrupting property of a chemical substance according to claim 9, wherein the nucleic acid detecting probe is immobilized on a substrate for a probe-immobilized chip.

12. A method of detecting the endocrine disrupting property of a chemical substance according to claim 11, wherein the substrate includes an electrode, and the probe-immobilized chip is a probe-immobilized chip which electrochemically detects binding of a target nucleic acid to the nucleic acid detecting probe.

13. A method of detecting the endocrine disrupting property of a chemical substance, comprising:
(a) making the chemical substance act on a specimen;
(b) after the chemical substance has acted, obtaining a specimen nucleic acid from the specimen;
(c) obtaining an amplified product of the specimen nucleic acid using 2 or more kinds of nucleic acid detecting primers as defined in claim 6 and a nucleic acid amplifying enzyme;
(d) obtaining an extent of expression of the target nucleic acid by analyzing the amplified product obtained in (c); and
(e) detecting the endocrine disrupting property of the chemical substance by comparing an extent of expression of the target nucleic acid obtained in (d) with an extent of expression of the target nucleic acid when the compound has not acted on a specimen.

14. A method of detecting the endocrine disrupting property of a chemical substance according to claim 13, wherein the specimen nucleic acid is a mRNA, and the nucleic acid amplifying enzyme in (c) is a DNA polymerase having a reverse transcription activity.

15. The method of detecting the endocrine disrupting property of a chemical substance according to claim 13, wherein the specimen nucleic acid is a mRNA and, prior to amplification in (c), the specimen nucleic acid obtained in (b) is reverse-transcribed using a (dT) primer and a reverse transcription enzyme, and the resulting reverse transcription product is amplified as in (c) to obtain the amplified product.

16. A peptide encoded by a nucleotide sequence selected from a group consisting of nucleotide sequences described in SEQ ID No. 1, SEQ ID No. 3, SEQ ID No. 15 and SEQ ID No. 23.

17. A peptide described in an amino acid sequence selected from a group consisting of amino acid sequences described in SEQ ID No. 2, SEQ ID No. 16 and SEQ ID No. 24.

18. An antibody recognizing the peptide as defined in any one of claims 16 and 17.

19. A probe-immobilized chip comprising a substrate, and at least one probe selected from a group consisting of an antibody as defined in claim 18 which are solid-phased on the substrate.

20. A method of detecting the endocrine disrupting property of a chemical substance, comprising:
(a) making the chemical substance act on a specimen;
(b) after the chemical substance has acted, obtaining a specimen sample from the specimen;
(c) making the specimen sample react with at least one detecting probe comprising an antibody selected from a group consisting of an antibody as defined in claim 18;
(d) detecting the presence of the target substance by detecting binding of the detecting probe with a target substance; and
(e) detecting the endocrine disrupting property of the chemical substance by comparing the detection results obtained in (d) with the results of detection of the presence of the target substance when the compound has not acted on a specimen.


SEQUENCE LISTING (28 sequences)

SEQ ID NO: 1 - DNA, 2560 bp, Mus musculus
cggacgcgtgggcttgatgcccactgtgaggtggcagttaactggtatgctccacttgtagcccccatatctaaggacagaaccatttgcactgtgccgattatagatgtcataagcggcaacacatatgaaattataccccaagggggtggtgatgaagatgggtatgcccgaggagcatgggactggagtatgctctggaaacgggtgcctctgacctctcgagagaagagactgagaaagacaaaaactgagccgtatcggtctccagctatggcgggtggattgtttgccatagagaaggacttcttcttcgaactgggtctctatgatcctggtctccagatctggggtggtgaaaactttgaaatttcatacaagatctggcagtgcggtggcaaattgttatttgtgccttgttctcgtgttgggcacatctaccgtcttgagggctggcaaggaaaccccccacccctttacgttggctcctctccaactctgaagaattatgttagagtcgtggaagtctggtgggatgaatataaagactacttctatgctagccgtcctgagtcaaaggcgctgccctacggggacatatctgagctgaagaaatttcgagaagatcacaactgcaaaagtttcaagtggtttatggaagaaatcgcttatgacatcactgcccactaccctttgccccccagaaatgtcgagtggggtgaaatccgaggcctcgaaactgcatactgtattgatagcatggggaagacgaatggaggcttcgtggagctaggaccctgccacaggatgggtgggaaccagcttttccgaatcaatgaagcaaaccagctcatgcagtacgaccagtgtttgacaaaggggcctgatggatccaaagtcatgatcacacactgtaacctaaatgaatttaaggaatggcagtacttcaagagcctgcacaggtttacgcacatcacttccggaaagtgcttagatcgatcggaggtcctgcatcaagtgttcatctccacctgtgactccagtaaaatgactcagaagtgggagatgaataacatccacagtgtttgacaagaacagaggaaaccaacaatctacctactgacaagtacatttatggaggactgaaaaccgcctggaacctgctgcaaccattattactaattttgtacagctccaaacctggaacctctctgatcagttggaaggggcattgataaactgtgattttacaataacattatcatctgcagtgactgtttacaaaactgctcttaccttaaactctagatgtttacatcgttttttgttttgttttatgatgatgttggtaatttgtgcctttaactcggtttcctgaaccgccgagttaaagcatgtgttgtcttctttgggaatacactcaggggtctggaaggcagtttggttttttttgttgttgatgatgttgttcttgttttttaacacacttgaaaaaaaaaaaaggttggagtaagcagactttcacatccgacttggtgatgatcaacctgctgtgtatttaattttacatctttcggaagcactgccacgggtcgttggccagggtggccttccttcagttacgctgctgtttgaaaggtgaatttcaacacatttagtgcctctttcatttctcagtatattgtttgagagcttctagtgacacttctatgatggtgacaatgaatgtcacctggggaacccgtctgttactcacaggagaatttcttggctatgaagtggaatgttgtatggctgggtcgaggacaacagtgggcccaactcaaggctgttccgtggggcttgaagattctgtggcattacctccctggcctctgttcacaccagcttctaggctagcaaaggagtccttcttctaaaaggaggggttggtttttgcccatctacattttgcatctgctttccccagtaccagtcatagaaaactaaacataattctcagtttgaaacttgaaggggggttgaggggcagggaaacaacacaataaagccctagtgtggacgccttaggttggtcctgaggtaaaaatccccaagcccttgtgagatggaagccttagagaaccacctcgaagcacaagctgtgcacagagataaactgacactttgtctacattcaaaggaaattccatgagcattctctttaatttaggaaaattagtatctaattttctattcttagatcttattatctaagaatacaaatcatcaacaaatgtggacgacattctgcgaagttagatgctagctctgtagggttccttaatttcttttttaagaagtataaattttttttatattccccagggaaaagaagatttaatttgaacatttattaatacaatacctactttaagagaaccaaatgttgaaaactttaatttctaggaagtctcttattacaccccccttcccccaagataaaatgtctacattgagtgctaaaaaaaaaaaaaaaaaaaaaaaaaaaaaaaaaaaaaaaaaaaaaaaaaaaaaaaaa

SEQ ID NO: 2 - protein, 362 aa, Mus musculus
RTRGLDAHCEVAVNWYAPLVAPISKDRTICTVPIIDVISGNTYEIIPQGGGDEDGYARGAWDWSMLWKRVPLTSREKRLRKTKTEPYRSPAMAGGLFAIEKDFFFELGLYDPGLQIWGGENFEISYKIWQCGGKLLFVPCSRVGHIYRLEGWQGNPPPLYVGSSPTLKNYVRVVEVWWDEYKDYFYASRPESKALPYGDISELKKFREDHNCKSFKWFMEEIAYDITAHYPLPPRNVEWGEIRGLETAYCIDSMGKTNGGFVELGPCHRMGGNQLFRINEANQLMQYDQCLTKGPDGSKVMITHCNLNEFKEWQYFKSLHRFTHITSGKCLDRSEVLHQVFISTCDSSKMTQKWEMNNIHSV

SEQ ID NO: 3 - DNA, 942 bp, Mus musculus
  misc_feature (2)..(2): n = a, c, g, or t
gnccagtaccagtcatagaaaactaaacataattctcagtttgaaacttgaaggggggttgaggggcagggaaacaacacaataaagccctagtgtggacgccttaggttggtcctgaggtaaaaatccccaagcccttgtgagatggaagccttagagaaccacctcgaagcacaagctgtgcacagagataaactgacactttgtctacattcaaaggaaattccatgagcattctttttaatttaggaaaattagtatctaattttctattcttagatcttattatctaagaatacaaatcatcaacaaatgtggacgacattctgcnaagttagatgctanctctgtanggttccttaattncttttttaanaantataaattttttttatattccccangnaaaanaaaatttaatttnaacatttattaatacaatacctactttaacaaaaccaaatnttnaaaactttaatttctaaataaanctcttattacacccccccttccccnaatataaaatttcctcnttaantnccatatnnnnactcnccttnnccntctttttncatcctcctnncctctnntncnccnttncntnnnnccnnctcnctttttttncnccccnntnccntctnnncntcttnttnnctnnccnttnctccnntccccnnntttntctntattcntcntnctttncctctnttctcntncccnttntttctccctttccccttcnntncnntnctntnttttctcttcctcctnnctnntnncttnttcncttccncttttctcttcnctttncccntcctnnccctncccnncntnncctctnttctctntccctttnnctnccctcntcttctnncncntnctnctntctntnntncntctccttctctnctcnntcttcccnntctccncnnnttntccccn

SEQ ID NO: 4 - DNA, 678 bp, Mus musculus
  misc_feature (24)..(24): n = a, c, g, or t
aaatattattcatgttgtaccaanctagtatagtatgtatgttttgtgggtgtgtatatacgtaatatattttatatatanatanatatgagagagagngntttagtgactttgatacgggttggtgcaggtgantttattactgagccaaatgaggcacataccgagtcagcagttggagtccagggcattcagtgctgtgcatggttggcatacatgtacagtgcctatcccgtgcagtaagaatcaccaccggcatgccacatccagtactcagaactcagagccagcaatattttatttgtagacaatcagcttgaatctatagattttatgactggcaaatgattataatcctgaacccataacgctcgcaaaaggagacccagagcgtcttggagagacgtttggtttggttttgtgcctgggagtcagtcagtacaacagcaactgtatgtgcttatgttagtctcaagatccttaacttgttgaccttattattttggaaatttttttgtattatatttgtgggaaggtaaaataatgattttaagatttttatcaaatatggagattagttatttatgaaaaacaaagaaatgtctatttttgtttctttgttcccaattaatgtagataactttttaaaatgcattaaagcaatggtgaagaaaatgt

SEQ ID NO: 5 - DNA, 412 bp, Mus musculus
  misc_feature (2)..(2): n = a, c, g, or t
gnccagtactcagaactcagagccagcaatattttatttgtagacaatcagcttgaatctatagattttatgactggcaaatgattataatcctgaacccataacgctcgcaaaaggagacccagagcgtcttggagagacgtttggtttggttttgtgcctgggagtcagtcagtacaacagcaactgtatgtgcttatgttagtctcaagatccttaacttgttgaccttattattttggaaatttttttgtattatatttgtgggaaggtaaaataatgattttaagatttttatcaaatatggagattagttatttatgaaaaacaaagaaatgtctatttttgtttctttgttcccaattaatgtagataactttttaaaatgcattaaagcaatggtgaagaaaat

SEQ ID NO: 6 - DNA, 16 bp, Mus musculus
aaatgtggacgacatt

SEQ ID NO: 7 - DNA, 21 bp, Mus musculus
tttggttttgttaaagtaggt

SEQ ID NO: 8 - DNA, 21 bp, Mus musculus
agacaatcagcttgaatctat

SEQ ID NO: 9 - DNA, 18 bp, Mus musculus
aggtcaacaagttaagga

SEQ ID NO: 10 - DNA, 21 bp, Mus musculus
gaggtggcagttaactggtat

SEQ ID NO: 11 - DNA, 19 bp, Mus musculus
cgcactgccagatcttgta

SEQ ID NO: 12 - DNA, 21 bp, Mus musculus
tagtgactttgatacgggttg

SEQ ID NO: 13 - DNA, 21 bp, Mus musculus
cccaggcacaaaaccaaacca

SEQ ID NO: 14 - DNA, 271 bp, Mus musculus
  misc_feature (5)..(10): n = a, c, g, or t
ggagnnnnnncgtacaggtccagagccaagggtgaagacaaagacaaagcctcttgataggaaaaaaaaggcttgtgggctggagagatggctcagcggttaagagtacagactgctctaccagaggtcctgacttcaattcccagtaaccacatgttggcccacaaccatctataatgggatctgataccctcttctggtgtgtctgaagacagcaacagtgtactcacatacataaaacaaataaatatatcttaaaaaaaaaaaaaaa

SEQ ID NO: 15 - DNA, 4818 bp, Mus musculus
ggagcgccgaggccagtgttggacactttcccaccatgccgacaacagtttaaatatctgtcagctttctaaaagtttaactgaaggaaacctctaagacttcgtacctgaggaccaaagtgcagccaccaattatcagactttcaggatgaatctggaaaaactcagcaagcctgaactcctgacactgttcagtatccttgaaggagaacttgaagcaagagaccttgttatagaagctttaaaggcccaacacagagatactttcattgaagaacgctatggaaaatataacatcagtgaccctctaatggctcttcagagagattttgaaacactgaaggagaaaaatgatagtgagaagcagccagtgtgcacaaaccccctctctgtcctcaaggcggtgatgaagcagtgcaagaacatgcaggagcgcatgctgtcccagctggccgcagccgagagcaggcaccgcaaggtgatcctcgaccttgaagaagaaagacagaggcatgcacaggacactgctgaaggagatgatgtcacctacatgctggagaaggagagggagagactgacgcagcagttggaatttgagaagtcccaagtgaaaaagtttgaaaaagaacaaaagaagctgtccagtcagttggaggaggagcgcacccgccacaagcagctctcttccatgctggtgctcgagtgcaggaaggccactagcaaggcagccgaggaagggcagaaggccggagagctgagcctgaaactggacaaggagaagagccgggcgagcaagctagaggaagagttggcagccgagaggaagcggggcctgcagacggaggcccaggtggagaagcagctgtctgagttcgatatcgaaagggagcagctgagagccaaactgaaccgagaggagaaccggacccgtgccctgaaggaggaggtggagagcctgaagaagcttgtgaaagacctagaggcagcccagcagcaccgcagcaccagtgagcagggcagggagccagtgaccatgtccagaggcacagccacggagcctcccatgcgagtgtctgcgttttgtcaaacagagagtgttcagacagaaagaagccacgggagtgtcataaccaagctgacagacactggtcttccaggtcccaccacggctgcttactcatatgctaaagctaatggccactgtgacccagagatacagactaccagggagttgacctcagacagcagcacagaaaaccaagggcctccacgggagaaatctgcagtagcggctcaggagaaaccagtggagaatggtgggtgtcctgtaggaactgagactccagtcacgatgcctagtcacctcccttccagtggcagctccctgtctcccagcagcacagcctcttcctctctgacatcatctccttgctcctccccagttctaaccaagcgtctcttggggtcagcagccagcagcccgggctaccagtcttcctaccaagtcgggatcaaccagcgcttccatgccgctcggcacaaatttcagtcccaggcggatcaggatcagcaagccagcggtctccagagccctccatccagggacctgtcccctaccctgttagacaactctgctgccaagcagctggcccgaaacacagtcactcaggtgctctccagattcactaaccaagggccaattaagcccgtctctcccaacagttctccctttgggacagactaccgaaatctggccagcactgccagcccaagaggtgacaccagccattcacccactccagggaaagtgtccagccctctgagccccctctctccaggaatcaagtccccaaccatccccagagctgagagaggaaaccctccgccaatcccgcccaaaaagcctggcctcaccccttcgcagtctgccaccactccagtaaccaagactcattcccaggcatcctccctggccgccacagaagacctcgccagcagctgctcccccagtgccgtggtggccaatggcaaagatgttgagatacttttgcctaccagcagctagtccctagggctgagtctccacatttgacattccatcagactcgggccaagagctcagcgttaaaccctggagtcaaatcatgttatttatttgatagtagctacagccatctgtataatacattcagtgtatttacctttttgtatttttttaagtagaaattgaatactttggatttttatgactcacctctttgtactaagctagaaggggacctcatagacatagcgcatctcaagctctgcacttgccagcgtgctgcgacgaggcggctgactggggaccaggcggtgctactgccagtcggggagctagaattcattactcagcactcatttcaaattatgcagaagcggttcgtgcagatttttattccagatgaacatgttttaaaagtgcctgaaatcgcctgccacgggaatgtgattctgcggcagaaacaaaagacagagcatttcttgcagcaaatgagatcctctgtgaatctgaatgtcaaaaaaaccaacactgcttttaatcctctctgactgtttaatgccagctttgtgttctgaaccgaggtttgcataagtagagtggaaatccttctgaagggcgggaactctgtcaggctgagctctgtaaagccctcaagaaaccctccagaacacttagcagttggggtgctgattttcttgtgctttggaaaaccttaagtcattctccgtttcctgcatcacttcttgagtagtgaagtcacgtctccattcaaaccaaccctcaagcatcggccatgggaacttctgattctggttgcttttacttgaataggaaatgacataatgaactctttattctgtgatcaaaaagcaataacttaacaagtcagtctttgtaatactgtaagtcagaattatacagattttaccaaattgttacattcactctccatgctgccagcacttctatctatggaaccaaatgggcgtatatctgtatctgtatagtacacaccccttttacatgttcagcattctcacactcaagcacataagtattagtgcaattgtatctctggagcttgcagctgtagcataaaggacaaagtagaatcaatcgcatgttaagattacctaccaaagcaattagactgtgacttggagccagtcagtcaacaaagaaatccatttgacaaggaagtggacaatcccaaactaagctcacatcatcagcatcataggaaagcccattaaatcgtcctagtgccacagaactcagctagccatgtgagtgagtgagacagcacagcccctcaagtgctatccactcagccatagcatcctagccccgtgtaatggggccacccctttgagccccagctcctgtcatgcaaaaattgcagatggaggttaccaattctctaaacgttcatggaattgagattcccacaagaagattctcattctgattttaaatgttttttaaatgctgctgattagacaaacatggctgcaaatgactttagcttgcaggctaccaagctgcagcctctccctccctagaacctgtgctgtttcccttgtagtgagttgtacagacagaaacccccaggcttctccactgtgtcagctacatctgaccaagtccacagagcaggacagccttgctcttggcaagcagaacccccgacacaccagctcaggacactggttacacagttttttatccagagtaagagagaggaaggccttggtgtctttacataaattgttctggccagcatctacccagaagcccctgcactactgtaaccgtttatagggaaggagaatgggtgatcattggaaacacctgatcatgtttggcaaaaccaaaggattcagtgggcccattcagatgagtcacaaggaattagcaattgatgtccgtctggaaccacagagctgaaaacaaaaagtctccctaataatttagctaatagctcttaagtaaaattttattaaccctttgagtcagtgggcaagaggcaggtgattaaaatcttggtgggcaagtcaacaacagggtatactgttgactctccagcttacagtttaaatggttaaggtaaatgcatgacacatgaaagtgtggctggtgtctgctgaaatgatggagacatgtgaaactgtcccgagcgatcctgcatggaatttactcctattgcggacgcaaaatagaccagtgatgatcaaacgctgaaccttctcctcaggagagactgcagggcctcagtgttcttacctctcttgctttaaatggtaatcttaaaagctgtgttgtgctcttcctctgtttggagacagatatatatagagaaagagaaatgctataggggagtttatttcttgggggggatgcactgaacaatatttcttttacagtataatacaggggtgggatatgcaaaagaaatgtgtatttttgctagttcctatcttctgagataataagcacaatcctgaaatggatccaataattcagctagatattatagattagtatttcagtttgcagtagattgtgagtgtgtgcgctaagtaaaagttccatgattcacaatttgggtattaaccatatcaaagaagctgttactggtcagcagaatctaatgatgtgctcggtgtaacacaagcgttaactaagtcattaacatttgtaaggccaagtgcagaagtcttcatttttatagtagactacattaaaacaagttaaatcgcaaaaaaaaaaaaaaa

SEQ ID NO: 16 - protein, 600 aa, Mus musculus
MNLEKLSKPELLTLFSILEGELEARDLVIEALKAQHRDTFIEERYGKYNISDPLMALQRDFETLKEKNDSEKQPVCTNPLSVLKAVMKQCKNMQERMLSQLAAAESRHRKVILDLEEERQRHAQDTAEGDDVTYMLEKERERLTQQLEFEKSQVKKFEKEQKKLSSQLEEERTRHKQLSSMLVLECRKATSKAAEEGQKAGELSLKLDKEKSRASKLEEELAAERKRGLQTEAQVEKQLSEFDIEREQLRAKLNREENRTRALKEEVESLKKLVKDLEAAQQHRSTSEQGREPVTMSRGTATEPPMRVSAFCQTESVQTERSHGSVITKLTDTGLPGPTTAAYSYAKANGHCDPEIQTTRELTSDSSTENQGPPREKSAVAAQEKPVENGGCPVGTETPVTMPSHLPSSGSSLSPSSTASSSLTSSPCSSPVLTKRLLGSAASSPGYQSSYQVGINQRFHAARHKFQSQADQDQQASGLQSPPSRDLSPTLLDNSVAKQLARNTVTQVLSRFTNQGPIKPVSPNSSPFGTDYRNLASTASPRGDTSHSPGTPSQSATTPVTKTHSQASSLAATEDLASSCSPSAVVANGKDVEILLPTSS

SEQ ID NO: 17 - DNA, 19 bp, Mus musculus
acaaagcctcttgatagga

SEQ ID NO: 18 - DNA, 21 bp, Mus musculus
agacacaccagaagagggtat

SEQ ID NO: 19 - DNA, 20 bp, Mus musculus
attgtgagtgtgtgcgctaa

SEQ ID NO: 20 - DNA, 21 bp, Mus musculus
gacttctgcacttggccttac

SEQ ID NO: 21 - DNA, 21 bp, Mus musculus
ctctgtcctcaaggcggtgat

SEQ ID NO: 22 - DNA, 21 bp, Mus musculus
gagctgcttgtggcgggtgcg

SEQ ID NO: 23 - DNA, 2896 bp, Mus musculus
gtgtttgttcctgcggaggagccggcgccatggcggttctcttggagaccactctgggcgacgtggtcatcgacttgtacactgaagagcgtccccgggcttgcttgaattttttgaagctgtgcaaaataaaatattacaactattgcctcatacacaatgtacagagggattttatcatacaaacaggagatcccacggggactggccgcggaggagagtctatatttggacaactgtatggtgatcaagcaagcttttttgaagcagaaaaagtaccaaggataaagcacaagaagaaaggcactgtgtccatggtgaacaacggcagtgaccagcacgggtctcagtttcttatcactacaggagaaaacttagattaccttgacggtgttcatacagtgtttggtgaagtgacagaaggcatggacatagttaagaaaatcaatgagacctttgtggacaaggattttgtaccatatcaagatataaggataaatcatacagtgattttagatgatccatttgatgaccctcctgatttattaattcctgatcgatcacccgagcctacaaaggaacaattagatagtggtcgaataggagcagatgaagaaattgatgacttcaaaggaagatcagctgaagaagtagaagaaataaaggcagaaaaagaggctaaaactcaagctattctcttggagatggtgggagacttacctgatgcagatattaaacctccagaaaatgtgctgtttgtgtgtaaattgaatccagtgaccacagatgaggacttggagataatattctcaagatttgggccaataagaagttgtgaagttatccgggattggaaaacaggagagtccctctgttatgcttttattgaatttgaaaaggaagaagactgtgagaaagcattcttcaaaatggataatgtgcttatagatgacaggaggatacatgtggatttcagccagtctgttgcaaaggttaaatggaaaggaaaaggtgggaaatataccaagagtgatttcaaggagtatgaaaaggaacaggataaaccagccaatttggttctgaaagaaaaagtaaagcccaaacaggatgcaaaatatgatcttatactagatgagcagggagaagactccaaatcaagtcactcacacacaagtaaaaagcacaaaaagaaaacacgccactgctccgaagaaaaagaagatgaagagtacatgccaatcaaaaatcctaatcaggatatctacagggaaatgggttttggtcattatgaagaagaagaaagctgttgggagaaacagaagaatgaaaagagagaccgacgtcagaaccgcagtcgtagccggtctcgagaaagggatggtcactatagcaacagccacaaacccaagtaccagacagaaccttatgagagggaaagaagcagaaagagggacagaagcaggagtccaaagaaatccaaagctaaagaaaaatccaagtacagatgagcagaggaggtggacaattgtagcaggtctgctccggcctgagctggggcacgcagacgcactcagtgcctgccagcacagctgactgcggtttcgatgttagcgttgttgcctttcaggttacactgatggtgtggggctctggaagagagggaggcattttgtgtatactttttatacaaattttattgttgtgcatacatggtaacgttcatatttgacatctttcatagtttcagtcttttaagaatgaatcatttcatatcacagaagtccataaacttgtatgtaaagcataaaacagaaaatgtacctggctgcagcgcaagaggtaagagtgtcactcactatgaaagctccatatatcccttctccttgcaggaattattctgagttgtgtttaccctgctgttaattttgtatgtttttgaattttatacaaatggcacaatgtatattttccttctgtgactttctttttttaagagtccatttatttttattgttacatagttgtgaatgtactacgcttgatctgttgatggacttttaggttgtttgttagtttttgctatctgaggaatgccactgtgaacatttcaactgagcatggcactttctggccatgtggtttgagttttcaatgcatttatagttatggttgctgtaatttacctggtatgtgaattgtatggggtcaggggtagccacaagagtagttagtctaccatacagctcagagcacatttgctccattattgtacatagttttgactaccaatagaaaaggcttaactatatcaggagctagcatcttttctagggagatggctcagtgggtacagtgtttcctcatcagcacaggactgagttcagatcctcaccacatacccagtgcatgcgactaataatccagtactggggagacaggagcgggggaagctccaggcagttcgagtgaccgaggaagacactcggcactgacctcttgcctccacatactgtacaaactcgtgcgtgcaggcacacacacacccgaatacacagctgttctgcatacagaacggtaggtgttgctcacacagatgaacctaaaacatggtctagaagaatgagcagggacaggctggaagcaacgagaagtcatccactgactgcggagactcgatttaagagctctggagtattgaaataggaaaatgtatgcaccaagcgtttcaaggaaaacatatccttgattccaaatgataagaaaccgttttatttttcctatgtatttgacagtaactatatgcttatgggagtgtaatatgtttgctatatgttaatattctggaacaagtaaatccatttgcagacatatcagcttacttttttctttcttttgg

SEQ ID NO: 24 - protein, 493 aa, Mus musculus
  MISC_FEATURE (24)..(25): X = any amino acid
MAVLLETTLGDVVIDLYTEERPRXXCLNFLKLCKIKYYNYCLIHNVQRDFIIQTGDPTGTGRGGESIFGQLYGDQASFFEAEKVPRIKHKKKGTVSMVNNGSDQHGSQFLITTGENLDYLDGVHTVFGEVTEGMDIVKKINETFVDKDFVPYQDIRINHTVILDDPFDDPPDLLIPDRSPEPTKEQLDSGRIGADEEIDDFKGRSAEEVEEIKAEKEAKTQAILLEMVGDLPDADIKPPENVLFVCKLNPVTTDEDLEIIFSRFGPIRSCEVIRDWKTGESLCYAFIEFEKEEDCEKAFFKMDNVLIDDRRIHVDFSQSVAKVKWKGKGGKYTKSDFKEYEKEQDKPANLVLKEKVKPKQDAKYDLILDEQGEDSKSSHSHTSKKHKKKTRHCSEEKEDEEYMPIKNPNQDIYREMGFGHYEEEESCWEKQKNEKRDRRQNRSRSRSRERDGHYSNSHKPKYQTEPYERERSRKRDRSRSPKKSKAKEKSKYR

SEQ ID NO: 25 - DNA, 20 bp, Mus musculus
aataggaaaatgtatgcacc

SEQ ID NO: 26 - DNA, 21 bp, Mus musculus
tagcaaacatattacactccc

SEQ ID NO: 27 - DNA, 348 bp, Mus musculus
gatacaggggtgggatatgcaaaagaaatgtgtatttttgctagttcctatcttctgagataataagcacaatcctgaaatggatccaataattcagctagatattatagattagtatttcagtttgcagtagattgtgagtgtgtgcgctaagtaaaagttccatgattcacaatttgggtattaaccatatcaaagaagctgttactggtcagcagaatctaatgatgtgctcggtgtaacacaagcgttaactaagtcattaacatttgtaaggccaagtgcagaagtcttcatttttatagtagactacattaaaacaagttaaatcgcaaaaaaaaaaaaaaa

SEQ ID NO: 28 - DNA, 280 bp, Mus musculus
  misc_feature (2)..(2): n = a, c, g, or t
gncatggtccataanaatgagcagggacaggcnggangcnacnanaangcntccactgnctgcggngactcnantcaanagctntggngnatngaaataggaaaatgtatgcaccaagcgttncaaggaaaacatatccttgattccaaatgataagaaaccgttttatttttcctatgtatttgacagtaactatatgcttatgggagtgtaatatgtttgctatatgttaatattctggaacaagtaaatccatttgcagncatancaaaaaaaaaaa